(19) United States
(12) Patent Application Publication
(10) Pub. No.: US 2016/0113709 A1
(43) Pub. Date: Apr. 28, 2016

(54) MYOCARDIAL ABLATION BY IRREVERSIBLE ELECTROPORATION

(71) Applicant: TEL HASHOMER MEDICAL RESEARCH INFRASTRUCTURE AND SERVICES LTD, Ramat-Gan (IL)

(72) Inventor: Elad MAOR, Tel-Aviv (IL)

(21) Appl. No.: 14/894,349

(22) PCT Filed: Mar. 5, 2014
(86) PCT No.: PCT/IL2014/050219

§ 371 Date: Nov. 26, 2015

Related U.S. Application Data
(60) Provisional application No. 61/956,283, filed on Jun. 5, 2013.

(51) Int. Cl. A61B 18/14

(52) U.S. Cl. CPC A61B 18/1492; A61B 2018/00267

(57) ABSTRACT

Selective cellular ablation by electroporation, applicable, for example, to bulk tissue in the beating heart. Protocol parameters potentially induce tissue loss without thermal damage. Device and method are potentially applicable for myocardial tissue ablation to treat arrhythmias, obstructive hypertrophy, and/or to generate natural scaffolds for myocardial tissue engineering.

### RELATED APPLICATIONS

This application claims the benefit of priority under 35 USC §119(e) of U.S. Provisional Patent Application No. 61/956,283 filed Jun. 5, 2013, the contents of which are incorporated herein by reference in their entirety.

### FIELD AND BACKGROUND OF THE INVENTION

The present invention, in some embodiments thereof, relates to the field of tissue decellularization by electroporation, and more particularly, to non-thermal ablation of cellular components in the beating heart.

Hypertrophic cardiomyopathy (HCM), a common genetic cardiovascular disease, may lead to heart failure, for example, to hypertrophic obstructive cardiomyopathy (HOCM). Heart failure which is drug refractory may result in a recommendation for surgical myectomy. However, surgery is unsuitable for high perioperative risk patients, and the surgical technique is confined to major medical centers with substantial experience with the procedure. Non-surgical alcohol septal ablation is a treatment alternative available for some high-risk patients, but use is not widespread.

Electroporation has been used extensively for in vitro gene transfer, and in drug delivery, for example electrochemotherapy. During electroporation, microseconds-long direct current electric pulses applied across a cell membrane create aqueous pores which increase its permeability. Increased permeability comprises the creation of aqueous pores across the cell membrane, allowing free transmembrane exchange of ions and large molecules.

For certain electroporation pulse parameters, the increase in membrane permeability is transient, and the cells affected by the pulses can survive the electric insult. Non-thermal Irreversible Electroporation (NTIRE), however, comprises the use of electroporation parameters which induce cell death by creating pores in cell membranes sufficient, for example, to induce irrecoverable loss and/or imbalance of intracellular components. Ablation, for example of solid tumors, is induced within microseconds, without generation of heat that could potentially damage extra cellular components.

### SUMMARY OF THE INVENTION

According to an aspect of some embodiments of the present invention, there is provided a method of reducing a volume of myocardial tissue in a mammalian heart by exposure to an electrical field, comprising: positioning an electrode array comprising at least a current source electrode and a current sink electrode to select a target bulk comprising the myocardial tissue; and delivering a pulsed electrical field through the electrode array to the target bulk, electroporating cells therein in a continuous volume extending between the current source electrode and the current sink electrode; the electroporation of cells leading to reduced volume of myocardial tissue in the target bulk.

According to some embodiments of the invention, the reducing comprises death of cells within the bulk of myocardial tissue.

According to some embodiments of the invention, the death of cells within the bulk of myocardial tissue comprises death after irreversible electroporation of cellular membranes.

According to some embodiments of the invention, thermal heating due to the pulsed electrical field is below a threshold of thermal damage to the intracellular matrix within the target bulk.

According to some embodiments of the invention, the threshold of thermal damage is 55° C. or lower.

According to some embodiments of the invention, the reducing occurs without scarring due to damage by ohmic heating.

According to some embodiments of the invention, the positioning comprises inserting the electrode array into a lumen of the mammalian heart.

According to some embodiments of the invention, the target bulk comprises a portion of the wall of the left ventricle of the heart.

According to some embodiments of the invention, the target bulk comprises a portion of the subaortic ventricular septum.

According to some embodiments of the invention, the target bulk comprises a portion of the left ventricular free wall.

According to some embodiments of the invention, the target bulk extends over from between 1-9 cm² of a wall region of the heart, to a depth within the tissue of at least 1 mm.

According to some embodiments of the invention, the pulsed electrical field comprises a peak field strength above 250 V/cm extending continuously between the source electrode and the sink electrode.

According to some embodiments of the invention, the pulsed electrical field is delivered in pulses sufficiently short to remain below a threshold of thermal damage to the myocardial tissue.

According to some embodiments of the invention, the threshold of thermal damage comprises reaching a threshold temperature equal to 55° C. or lower.

According to some embodiments of the invention, the pulses are less than 200 microseconds in length.

According to some embodiments of the invention, the pulsed electrical field is delivered in pulses at a sufficient interval to avoid cumulative thermal buildup to a threshold of thermal damage.

According to some embodiments of the invention, the sufficient interval is at least 250 milliseconds.

According to some embodiments of the invention, the threshold of thermal damage comprises reaching a threshold temperature equal to 55° C. or lower.

According to some embodiments of the invention, the electrode array comprises at least three electrodes.

According to some embodiments of the invention, electrodes of the electrode array are activated at least partially asynchronously during the delivery of the pulsed electrical field.

According to some embodiments of the invention, the positioning comprises pressing by an electrode deployment mechanism to urge the electrode array toward the target bulk of myocardial tissue.

According to some embodiments of the invention, the pressing by the electrode deployment mechanism comprises expansion thereof.

According to some embodiments of the invention, the electrode deployment mechanism, when so expanded, occludes less than 50% of the lumen of the heart.

According to some embodiments of the invention, the electrode deployment mechanism, when so expanded, reduces flow rate through the atrial valve of the heart by less than 50%.

According to some embodiments of the invention, the expansion comprises outward pressing by a balloon against the electrode array.

According to some embodiments of the invention, the expansion comprises expansion of a metal framework carrying the electrode array.

According to some embodiments of the invention, the expansion is to a relative positioning of electrodes having inter-electrode spacings within 10% of a predetermined relative positioning.

According to an aspect of some embodiments of the present invention, there is provided an apparatus for reducing a volume of myocardial tissue in the wall of a mammalian heart, comprising: a plurality of electrodes comprising a current source electrode and a current sink electrode; the plurality of electrodes being disposed on the distal end of a catheter and insertable to the heart thereby; a voltage source, configured to deliver a predetermined electrical potential to the plurality of electrodes when deployed in the heart; and the plurality of electrodes being deployable within the heart to assume positions against the wall and predetermined relative to each other; wherein the deployed positions define a volume by the electrical field produced upon delivery of the electrical potential, the volume extending continuously between the current source electrode and the current sink electrode, and being comprised in a bulk of myocardial tissue—the myocardial tissue being comprised in the wall—which would undergo irreversible electroporating ablation upon delivery of one or more pulses of the electrical potential.

According to some embodiments of the invention, the reducing comprises loss of tissue volume within the bulk of myocardial tissue due to irreversible cellular membrane electroporation therein.

According to some embodiments of the invention, the loss of tissue volume is at least 50% of the volume of the bulk of myocardial tissue.

According to some embodiments of the invention, the electrical field induces ablation of cells within the bulk of myocardial tissue without destruction of the cellular matrix of the bulk of myocardial tissue by ohmic heating.

According to some embodiments of the invention, the electrical field induces ablation of cells within the bulk of myocardial tissue without thermal damage to non-ablated cells adjacent thereto.

According to some embodiments of the invention, the bulk of myocardial tissue comprises tissue of the left heart ventricle.

According to some embodiments of the invention, the bulk of myocardial tissue comprises tissue of the subaortic septum.

According to some embodiments of the invention, the bulk of myocardial tissue overlies a region of heart wall thickening due to hypertrophic cardiomyopathy.

According to some embodiments of the invention, the bulk of myocardial tissue overlies a region of heart wall thickening due to cardiac hypertrophy.

According to some embodiments of the invention, the apparatus comprises a balloon inflatable to urge the plurality of electrodes toward the bulk of myocardial tissue, and to distance them to predetermined relative positions, while the distal end of the catheter is inserted into the heart.

According to some embodiments of the invention, the balloon comprises a hollow oriented for the passage of blood thereinto when the balloon is inflated in the lumen of the heart.

According to some embodiments of the invention, the apparatus comprises a frame expandable to urge the plurality of electrodes toward the bulk of myocardial tissue, and to distance them to predetermined relative positions, while the distal end of the catheter is inserted into the heart.

According to some embodiments of the invention, the apparatus comprises a sheath member positioned radially over the expandable frame in a collapsed position, wherein the expandable frame is exposable to allow expansion thereof by axial displacement relative to the sheath member.

According to some embodiments of the invention, the bulk of myocardial tissue has extent along the wall which covers at least 50% of the area between the current source electrode and the current sink electrode where they are deployed to contact the wall.

According to some embodiments of the invention, the electrical field within the bulk of myocardial tissue comprises a field region having a maximum field strength above 250 V/cm during a period when the electrical potential is received.

According to some embodiments of the invention, the field region has everywhere within the bulk of myocardial tissue a maximum field strength above 250 V/cm during the period.

According to some embodiments of the invention, the bulk of myocardial tissue extends over from between 1-9 cm² of a wall of the heart, to a depth within the tissue of at least 1 mm.

According to some embodiments of the invention, the bulk of myocardial tissue is adjacent to at least one valve of the heart.

According to some embodiments of the invention, the electrical potential is deliverable to the electrode array in pulses, and the length of the pulses is sufficiently brief that thermal damage to non-electroporated cellular tissue does not occur.

According to some embodiments of the invention, a period when the electrical potential is delivered comprises electroporating pulses of from 20-200 μsec, separated by intervals of 250-2000 msec.

According to some embodiments of the invention, the whole period during which the electrical potential is delivered in one or more pulses is less than 10 seconds.

According to some embodiments of the invention, the plurality of electrodes comprises at least three electrodes.

According to some embodiments of the invention, the apparatus comprises a switching mechanism for directing the application of voltage potential to the plurality of electrodes, configured such that the at least three electrodes are actuatable to receive the electrical potential at least partially asynchronously from one another.

According to some embodiments of the invention, at least a portion of the bulk of myocardial tissue is subjectable to an electroporating electrical field delivered from at least two sets of electrodes differing in at least one member during a period when the electrical potential is received.

According to some embodiments of the invention, the apparatus comprises a radio-opaque marker positioned in the distal portion of the catheter, such that it indicates a rotational position of the electrode array when radiographically visualized in the lumen of the heart.

According to some embodiments of the invention, the apparatus comprises a thermal sensor disposed near the position of at least one electrode of the plurality of electrodes.

According to some embodiments of the invention, at least one parameter of the electrical field is variable based on a reading of the thermal sensor.

According to some embodiments of the invention, a parameter of the electrical field is automatically adjustable during ablating of the bulk of myocardial tissue, based on an electrical property monitored during previous delivery of electrical potential to the bulk of myocardial tissue.

According to some embodiments of the invention, the previous electrical potential is non-electroporating.

According to some embodiments of the invention, the parameter is at least one of a pulse duration, a pulse interval, a voltage, and a selection of an electrode.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a schematic drawing of a heart illustrating regions of hypertrophic muscle tissue comprised in the ventricular septum and/or left ventricular free wall, according to some exemplary embodiments of the invention;

FIG. 1B is a schematic drawing of the heart of FIG. 1A, into which an electroporating electrode array deployed from a catheter on a frame is inserted for ablation of a portion of the left ventricular free wall, according to some exemplary embodiments of the invention;

FIGS. 1C-1D are heart images illustrating regions of hypertrophic muscle tissue comprised in the ventricular septum and/or left ventricular free wall, according to some exemplary embodiments of the invention;

FIGS. 2A-2B are schematic views of an electrode array frame hinged on a catheter, and pressed into position for electroporating ablation of myocardial tissue by inflation of a balloon, according to some exemplary embodiments of the invention;

FIGS. 2C-2D are schematic views of a frame for electrode positioning, according to some exemplary embodiments of the invention;

FIGS. 2E-2F are schematic views of intercalating electrode pairs, according to some exemplary embodiments of the invention.

FIG. 3 is a schematic view of an electrode catheter in which the electrode array is pressable into position by a balloon shaped to allow the passage of blood therethrough when inflated, according to some exemplary embodiments of the invention;

FIG. 4A is a schematic view of a radially expandable electrode array comprising a pair of electrodes pressed into a deployed position for electroporating ablation of myocardial tissue by inflation of a balloon, according to some exemplary embodiments of the invention;

FIG. 4B is a schematic view of a radially expandable electrode array comprising multiple electrodes pressed into a deployed position for electroporating ablation of myocardial tissue by inflation of a balloon, according to some exemplary embodiments of the invention;

FIG. 5A is a schematic view of a radially expandable electrode array pressed into position for electroporating ablation of myocardial tissue by expansion of a frame, according to some exemplary embodiments of the invention;

FIG. 5B is a cross-sectional view of the electrode array and frame of FIG. 5A, held in a collapsed configuration by a sheath, according to some exemplary embodiments of the invention;

FIG. 5C is a schematic view of an electrode array and expandable frame, with exemplary dimensions thereof, according to some exemplary embodiments of the invention;

FIG. 5D is a flowchart of operations for electroporating ablation in a heart, according to some exemplary embodiments of the invention;

FIG. 5E is a flowchart of operations during a phase of electroporating pulse delivery in a heart, according to some exemplary embodiments of the invention;

FIG. 5F schematically illustrates a system for electroporating ablation of heart tissue, according to some exemplary embodiments of the invention;

FIGS. 5G-5I schematically illustrate variations on the shape and exposed regions of an electroporating electrode for use within a lumen of the heart, according to some exemplary embodiments of the invention;

FIGS. 6A-6D are photomicrographs showing decellularization by NTIRE of vascular smooth muscle cells of rodent carotid artery, illustrating general characteristics of NTIRE decellularization in cardiovascular tissue, according to some exemplary embodiments of the invention;

FIG. 6E is a graph showing effectiveness of decellularization by NTIRE of vascular smooth muscle cells of rodent carotid artery under a range of electroporation conditions, illustrating general characteristics of NTIRE decellularization in cardiovascular tissue, according to some exemplary embodiments of the invention;

FIGS. 7A-7D are photomicrographs showing decellularization by NTIRE of blood vessels in rabbit, illustrating general characteristics of NTIRE decellularization in cardiovascular tissue, according to some exemplary embodiments of the present invention;

FIG. 8 illustrates application of a needle electrode array to NTIRE ablation in a rat heart, according to some exemplary embodiments of the present invention;

FIGS. 9A-9F are photomicrographs showing decellularization by NTIRE ablation in a rat heart, according to some exemplary embodiments of the present invention;

FIGS. 10A-10B are graphs presenting echocardiographic results of NTIRE ablation in a mammalian heart, according to some exemplary embodiments of the present invention;

FIG. 11 shows a radially arranged array of electrodes usable for vascular cell ablation by electroporation, illustrating aspects of modeling to determine NTIRE parameters in cardiovascular tissue, according to some exemplary embodiments of the present invention;

FIGS. 12A-12B show a theoretical model of a radially arranged electrode array having the dimensions of the array of FIG. 11, with associated electrical field densities, illustrating aspects of modeling to determine NTIRE parameters in cardiovascular tissue, according to some exemplary embodiments of the present invention;

FIG. 12C shows predicted maximal and average temperature (° C.) as a function of time (seconds) due to 90 electroporation pulses with ΔV=600 volts, at a frequency of 4 Hz, using the model of FIGS. 12A-12B, illustrating aspects of modeling to determine NTIRE parameters in cardiovascular tissue, according to some exemplary embodiments of the present invention;

FIGS. 13A-13B schematically illustrate electroporating ablation from a region of heart wall, according to some exemplary embodiments of the invention; and

FIGS. 14A-14B schematically illustrate non-linear relationships between distance, voltage field strength, and electroporation, according to some exemplary embodiments of the invention.

### DESCRIPTION OF SPECIFIC EMBODIMENTS OF THE INVENTION

The present invention, in some embodiments thereof, relates to the field of tissue decellularization by electroporation, and more particularly, to non-thermal ablation of cellular components in the beating heart.

### Overview

An aspect of some embodiments of the invention relates to reduction of tissue volume by application of an electrical field to the myocardium. Reduction of tissue volume is a potential advantage, for example as a treatment for a heart diseased with hypertrophic cardiomyopathy (HCM) or cardiac hypertrophy. More particularly, an aspect of some embodiments of the invention relates to ablation by irreversible electroporation of a selectable bulk of myocardial tissue. Optionally, the bulk is of sufficient extent and depth that accompanying tissue volume reduction relieves interference with circulation by and/or through the heart. For example, tissue volume reduction relieves a blockage of a heart valve, decreases a pressure differential within or near a heart valve, increases a volume of a heart chamber, and/or increases the stroke volume of a heart chamber.

It is a potential advantage for treatment of HCM and/or cardiac hypertrophy to selectively remove tissue from hypertrophic regions of a heart, and particularly from within a lumen of the heart; for example, to remove tissue non-circumferentially, non-focally, and/or from a selectable bulk of tissue from of a wall of a heart lumen. In some embodiments, the therapeutic effect of the removal of said tissue bulk comprises the removal of a mechanical impediment to heart valve operation and/or blood flow. Surprisingly, the inventors have found that there exist electrode configurations and corresponding electroporation protocol parameters which remove a substantial (1-4 mm, for example) thickness of myocardial tissue from a heart wall, while allowing the heart itself to remain functional as a pump. Furthermore, this is possible with little or no thermal damage, with benefits, for example, to the acceleration of post-ablation healing.

In contrast to electroporating ablation targeted to modification of the electrophysiological properties of a heart, volume reducing electroporation, in some embodiments, uses electrode configurations and/or voltage pulse protocols which create irreversible electroporating conditions throughout a bulk of tissue large enough that its size itself potentially interferes with heart function. In some embodiments, this bulk extends for the full distance between two electroporating electrodes. In some embodiments, the bulk also retains substantial depth (1-4 mm, for example) between two electroporating electrodes. In some embodiments of the invention, this is achieved by concentrating of the electrical field to achieve a minimal field strength through a targeted volume. Source and sink electrodes are located close enough to one another, in some embodiments, that a voltage field created in a region between them remains above an electroporating threshold strength. In some embodiments, source (current flows out of) and sink (current flows into) electrodes are about the same in size, and the same and/or complementary in shape, such that the electrical field is similarly concentrated at both source and sink. Furthermore, in some embodiments, the working regions of each electrode are about equally distant from another electrode, for greater field uniformity. A potential advantage of greater field uniformity is that a larger targeted region can experience electroporating conditions, without forcing electrical field conditions in one or more parts of the target region to pass a threshold beyond which unacceptable thermal damage occurs.

In some embodiments, the bulk of tissue selected for volume reduction is a slab of myocardial tissue about 10-15 mm by 10-15 mm with a depth of about 1-3 mm. In some embodiments, the selected tissue bulk extent is about 8-12 by 17-23 mm, with a depth of about 2-4 mm. In some embodiments, the selected tissue bulk extent is about 20-25 by 20-25 mm, with a depth of about 8-11 mm. In some embodiments of the invention, the slab of myocardial tissue selected for volume reduction is at least 5 mm in a minimum dimension along the heart wall, at least 20 mm in an orthogonal direction along the heart wall, and at least 1 mm in depth. In some embodiments, the selected tissue bulk comprises a volume, the ablation extent of which extends continuously through the selected tissue bulk. In some embodiments, the total volume of the tissue bulk selected for ablation is about 100-300 mm³, 200-500 mm³, 400-1000 mm³, 800-1600 mm³, 1500-3000 mm³, 2000-4000 mm³, 3000-6000 mm³, 4000-8000 mm³, or another range of volumes having intermediate, larger, or smaller bounds. In some embodiments of the invention, the ratio of the ablated tissue bulk depth (for example, in mm) to the areal extent of the bulk across and along the heart wall (for example, in mm²) is about 1:100, 2:100, 4:100, 8:100, or another larger, smaller, or intermediate ratio. The three-dimensional extent of the myocardial tissue selected for ablation, in some embodiments, comprises a single continuous block of tissue. In some embodiments, the tissue selected for ablation comprises a bulk homologous in size and shape to tissue which could alternatively be removed to treat a hypertrophic cardiac muscle condition by surgical septal myectomy and/or by alcohol septal ablation. In some embodiments, the bulk of tissue selected for ablation comprises a volume of cardiac muscle sufficient to relieve blockage of a heart valve. In some embodiments, ablation comprises reduction of volume distributed throughout tissue in a targeted bulk, the reduction being about, for example, 40-50%, 45-65%, 60-80%, 70-90%, 90-95%, 100%, or another range of relative ablation volumes having the same, intermediate, larger or smaller bounds. In some embodiments of the invention, reduction of volume due to ablation is concentrated on removal of cellular components, while non-cellular tissue components are left relatively intact.

Upon electroporation of a tissue bulk, in some embodiments, contractile activity is immediately inactivated. Over a period following electroporation, typically a few days to a week (in some embodiments about, for example, twelve hours, a day, two weeks, or a greater lesser or intermediate period), the volume of the tissue bulk reduces. Tissue bulk reduction potentially comprises cells which are electroporated undergoing traumatic and/or apoptotic cell death, leading to clearance of their remains and reduction of volume in tissues of the heart.

In particular, an ablated region of tissue is comprised in a ventricular free wall and/or a ventricular septum. Additionally or alternatively, the region is a wall of the right ventricle. In some embodiments, electrodes are configured to ablate hypertrophic tissue selectively from such a region. Optionally, ablated hypertrophic tissue comprises tissue adjacent to valves of the heart (aortic and/or mitral valves). It is a potential advantage to remove tissue adjacent to valves, for reduction of static and/or dynamic forces tending to cause blockages of the valves (either by their mutual interaction, and/or by the ventricular wall itself).

In some embodiments, an electrode array is provided on a heart catheter, shaped and configured to direct electrical current to a selected region, and in particular, a slab-like region, of myocardial tissue within the lumen of the heart. In some embodiments, the electrode array is shaped to delineate the contours of another shape over the extent of the heart wall, for example, a circle, oval, filled arc, polygon, or another shape. In some embodiments, the outline of the shape of the tissue which is removed by electroporation along the extent of the heart wall follows the shape of the electrode array, offset from the electrode shape to an extent determined by the strength and/or shape of the electrical field which the electrode array produces.

An aspect of some embodiments of the invention relates to positioning a myocardial electroporation electrode array within a lumen of a beating heart.

In some embodiments of the invention, electrodes are pressed against myocardial tissue by inflation of a balloon attached to a microcatheter used to introduce the electrodes to the heart. In some embodiments, the electrodes are attached to the microcatheter by a hinge member. In some embodiments, the electrodes are self-expanding, or attached to one or more self-expanding members. For example, one or more members constructed from a shape-memory alloy such as Nitinol are used to press the electrodes against tissue when a restraining member is removed from a “basket” or other self-expanding structure. It is a potential advantage for the electrode array to be operable without preventing the functioning of the heart as a circulating pump during an electroporation procedure. In particular, it is a potential advantage for the electrode array to be pressed to the heart wall by a compliant structure, such that electrical contact is maintained during the cardiac beat cycle. Optionally, this is without prevention of pumping.

In some embodiments of the invention, the expanded and/or deployed electrode array and/or its supporting structure fills a heart chamber sufficiently that it is held in place by opposing pressures against the heart chamber walls and or the portal by which it passes into the heart. In some embodiments, the structure is compliant over a fractional shortening range of up to about, for example, 45%, 35%, 25%, 20%, 15%, 10%, or another larger, smaller, or intermediate fractional shortening range. The anatomical plane of the fractional shortening range is any anatomical plane, but is particularly, for example, a plane transverse to the heart. It is a potential advantage to maintain contact for an prolonged period during a cardiac cycle; for example, half the cycle, the whole cycle, or any lesser or intermediate period of the cardiac cycle. Prolonged contact potentially allows electroporating pulses to be delivered more frequently, and/or with a greater confidence of positioning and/or efficacy.

It is a potential advantage for the expanded structure of the ablation electrode positioning device to be relatively open to allow the passage of blood therethrough. For example, a balloon member is shaped with a hollow such that it is expandable within a heart chamber without fully occluding it. Additionally or alternatively, an electrode-carrying basket—formed, for example, by an expanding frame—is sufficiently open to allow free passage of blood therethrough when expanded. In some embodiments, the expanded structure of the electrode positioning device does not entirely fill the volume of a ventricle, such that some blood can pass around the device for pumping.

It is an aim, in some embodiments of the current invention, for ablation of heart tissue to occur throughout a selected volume. The volume is selected, in part, by the relative spacings of the electrodes, since, for example, the electrical field gradient (V/cm) will be larger for a small spacing than for a large one, for a particular applied voltage differential. In some embodiments of the invention, both supply and return electrodes are provided on a common physical carrier, such that the positions of the electrodes are held in a desired relative arrangement upon deployment. Potentially, this helps ensure that electroporation without thermal damage and/or arcing occurs according to the parameters designed for the electroporation apparatus and protocol. In some embodiments of the invention, the spacing of electrodes between which an electrical potential is applied is about, for example, 3-5 mm, 8-12 mm, 13-17 mm, 18-22 mm, 22-27 mm, or another distance range having the same, intermediate, larger, and/or smaller bounds.

An aspect of some embodiments of the invention relates to the time-varying control of electrical fields during electroporation for ablation of heart tissue.

In some embodiments, individual electroporating pulses are relatively brief compared to the overall treatment period. In some embodiments, individual pulses are about, for example, 100 μsec long, or 150 μsec long. In some embodiments, pulse durations are, for example, 20-50 μsec, 40-80 μsec, 50-100 μsec, 80-160 μsec, or another range of durations having the same, larger, smaller, and/or intermediate bounds.

In some embodiments, a pulse is delivered with a field strength of at least about 250 V/cm. In some embodiments, the average pulse field strength (in a targeted region) is about, for example, 250 V/cm, 500 V/cm, 750 V/cm, 1000 V/cm, 1500 V/cm, 1750 V/cm, 3500 V/cm, or another larger, smaller, or intermediate field strength. In terms of ranges, average field strength induced in targeted tissue regions is about, for example, 250-400 V/cm, 350-700 V/cm, 600-800 V/cm, 800-1200 V/cm, 1200-1800 V/cm, 1500-2000 V/cm, 3000-4000 V/cm, or another range having the same, larger, smaller, and/or intermediate bounds. Absolute voltage differences across electrodes are chosen, in some embodiments, to achieve a particular desired field strength according to the electrode geometry. The applied voltage differential is about, for example, 600 V, 700 V, 800 V, 900 V, 1000 V, 2000 V, or another higher, lower, or intermediate voltage. Current delivered is, for example, about 1 Amp, or a larger or smaller amperage, according, for example, to the voltage and resistance of the circuit including the heart tissue, any of which is potentially time-varying.

In some embodiments, the total number of pulses delivered is, for example, 90 pulses. In some embodiments, the number of pulses is 5-15 pulses, 10-20 pulses, 15-30 pulses, 20-40 pulses, 30-50 pulses, 40-80 pulses, 50-100 pulses, or another range of pulses having the same, lower, higher, and/or intermediate bounds. The total period over which electroporating pulses are delivered, in some embodiments, is several seconds, for example 10 seconds. In some embodiments, the electroporation pulse delivery period is 1-2 seconds, 3-7 seconds, 5-10 seconds, 8-13 seconds, 13-20 seconds, 18-25 seconds or another range of time having the same, smaller, larger, and/or intermediate bounds. In some embodiments of the invention, the frequency with which pulses are delivered is 1 Hz, 2 Hz, 4 Hz, 10 Hz, and/or 50 Hz. In some embodiments, the frequency of pulse delivery is, for example, 2-3 Hz, 2-5 Hz, 4-8 Hz, 5-10 Hz, 40-60 Hz, or another range of frequencies having the same, higher, lower, and/or intermediate bounds.

In some embodiments, effectiveness of electroporation for volume reduction is increased by delivery of an increased number of pulses. For example, a single pulse may not upset the intra/extracellular separation maintained by each cell in a targeted region enough to result in total cell ablation. Additionally or alternatively, a single pulse may only affect a subset of cells in a treated population.

In some embodiments, electrodes are activated to deliver electroporating voltage pulses, with pulse intensities, numbers, and intervals chosen to induce electroporation substantially without causing heat damage. In some embodiments, certain minimum field strengths, and corresponding pulse durations are chosen for effective induction of electroporation. However, an excess of electrical power delivered into heart tissue can result in thermal damage at regions of high field concentration, such as near the electrodes. Thermal damage potentially destroys structure, including extracellular components, blood vessels, and/or duct scaffolds, leading, for example, to increased healing time. It is therefore a potential advantage to distribute the heating effects of energy delivered during electroporation in time and/or in space. More particularly, avoiding heating effects potentially places an upper boundary on how rapidly pulses of a given strength and/or duration can be safely delivered to a particular electrode position. In some embodiments, electroporation ablation substantially without heat damage comprises electroporation ablation of cellular components which leaves the extracellular matrix intact, for example, sufficiently intact to serve as a scaffold for cellular regrowth. In some embodiments, electroporation ablation substantially without heat damage comprises ablation of cellular components, while leaving adjacent blood vessels and/or a functional or partially functional portion thereof intact. In some embodiments, electroporation ablation substantially without heat damage comprises electroporation ablation of cellular components wherein cells which die do so through loss of homeostasis, rather than due to thermal denaturation.

In some embodiments, parameters of electroporating potential delivery are varied according to sensed data. For example, efficacy of electroporation occurring during a pulse is potentially reflected in changes in conductivity which represent the opening of holes in cell membranes. Optionally, electroporating pulses are adjusted, for example, in strength, duration, and/or frequency, according to sensed changes in tissue conductivity. Optionally, electroporating pulses are adjusted, for example, in strength, duration, and/or frequency according to thermal sensing data, potentially reducing a possibility for induction of thermal damage.

In some embodiments of the invention, pulse delivery is synchronized to the heartbeat cycle of the heart. Since the beating of the heart potentially deforms the electrodes, it is a potential advantage to synchronize pulse delivery to one or more predetermined points in the heartbeat cycle to control electrical field distortions. Optionally, electroporating pulse parameters are varied depending on when in the heartbeat cycle pulse delivery occurs.

An aspect of some embodiments of the invention relates to multiple electrodes provided for alternating use during voltage pulse delivery.

In some embodiments of the invention, electrode activation comprises temporal patterning of applied voltage fields among separate electrodes and/or tissue regions. In some embodiments, patterned electrode activation distributes thermal effects spatially. For example, an array of 4, 6, 8, or 10 electrodes (or a greater, lesser, or intermediate number of electrodes) is pair-wise activated over an electroporating period. It is a potential advantage to shift the positions of electrical fields delivered during an electroporation protocol, such that heating near to electrodes (where temperatures potentially reach a maximum) is distributed across the tissue over time. In some embodiments, the inter-pulse period is reduced to take advantage of a greater spatial distribution of heating effects among electrode locations.

A potential advantage of an overall reduced period of electroporation is reduced effects of limiting blood flow during an ablation procedure. A positioned array of electroporating electrodes and/or supporting structures potentially prevent blood flow to an unsustainable degree. Blood flow prevention comprises, for example, interference with valve operation, interference with heart contractions, exclusion of blood from a portion of the heart volume, and or obstruction of the passage of blood.

An aspect of some embodiments of the invention relates to structures which guide the positioning and/or monitoring of a myocardial electroporation electrode array within a heart.

In some embodiments of the invention, electrodes and/or electrode positioning members themselves are radio-opaque, permitting radiographic observation during a procedure. In some embodiments, one or more radio-opaque markers are additionally provided to allow distinguishing the position of the electrodes. In some embodiments, temperature monitoring, such as a through a thermistor, is provided on and/or near electrodes. In some embodiments, temperature measurements are used to verify and/or control the delivery of electrical pulses to tissue.

Before explaining at least one embodiment of the invention in detail, it is to be understood that the invention is not necessarily limited in its application to the details of construction and the arrangement of the components and/or methods set forth in the following description and/or illustrated in the drawings. The invention is capable of other embodiments or of being practiced or carried out in various ways.

### Examples of Hypertrophic Heart Disease

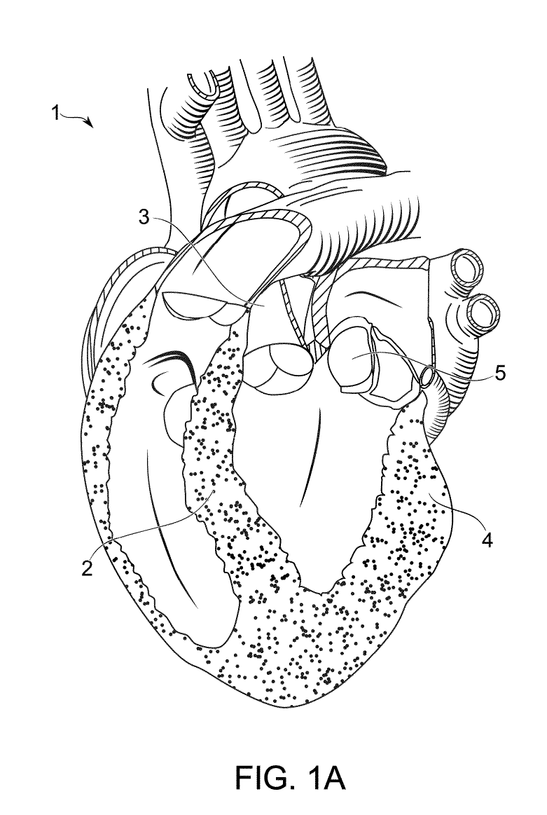

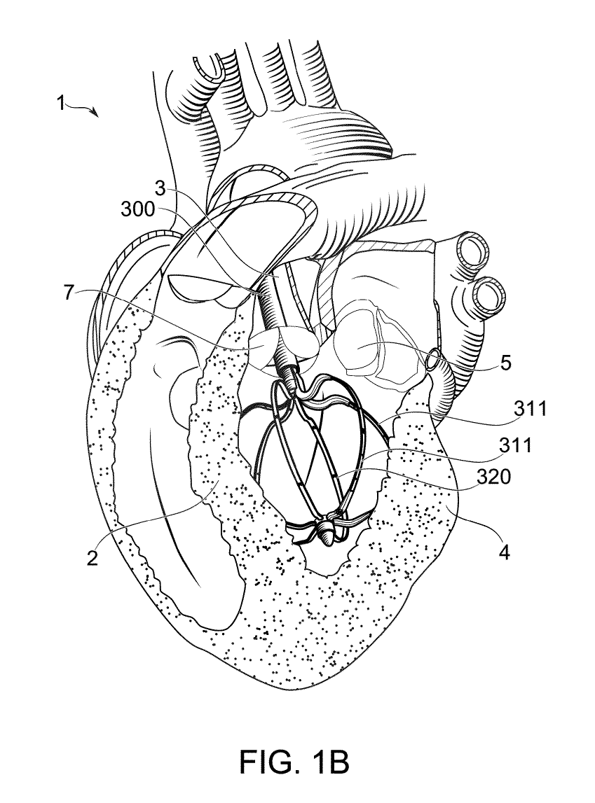

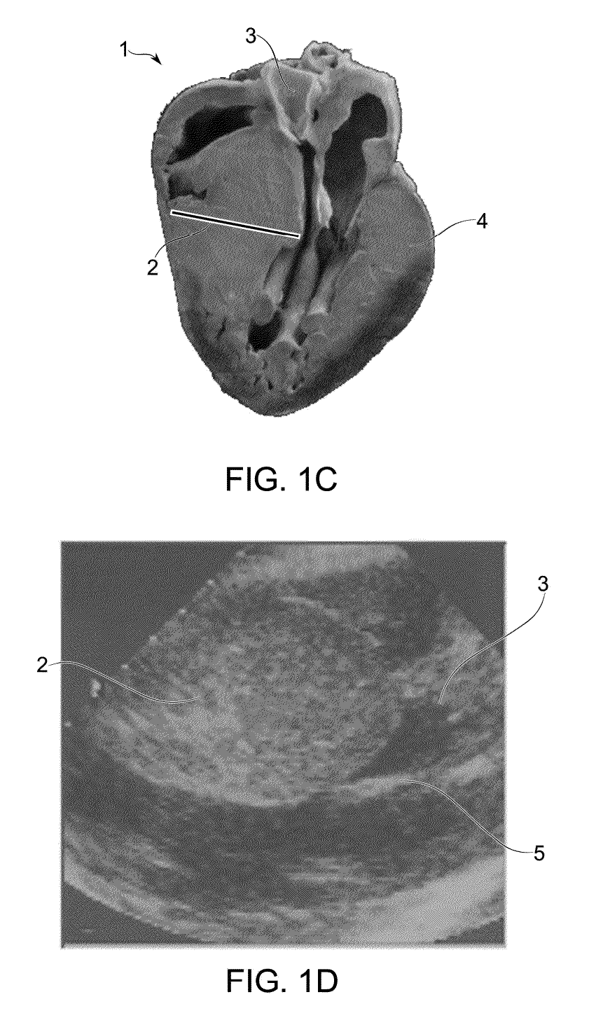

Reference is now made to FIGS. 1A-1D, which show views of heart 1, having regions of hypertrophic muscle tissue comprised in the ventricular septum 2 and/or left ventricular free wall 4. Hypertrophic tissue reduces heart chamber size, and potentially blocks flow (hypertrophic obstructive cardiomyopathy) leading to heart failure; for example, flow to the aorta 3 is potentially blocked via interference due to anatomical rearrangements from the valve comprising the anterior mitral leaflet 5.

FIG. 1B shows an electroporating and/or electrical field generating device 300 inserted into the left ventricle of heart 1 across the aortic valve 7, in a deployed configuration. In some embodiments of the invention, arms 320 (optionally comprising a basket-like arrangement like that of FIG. 1B) are orientable to position electrodes 311 in contact with and/or in proximity to a region of heart wall, for example, left ventricular free wall 4. Effective positioning of electrodes 311, in some embodiments, is such that the electrical field the electrodes generate during a voltage pulse permeates a selected bulk of myocardial tissue. During electroporation, a potential difference between electrodes 311 is generated, such that an electrical field is established therebetween. In some embodiments, the field gradient is sufficiently high within a bulk of heart tissue permeated by this field to induce electroporation of cellular membranes. The volume of heart muscle exposed to an electrical field, in some embodiments, is thereby potentially ablated and/or reduced, decreasing heart wall thickness, for example, to treat HCM. It should be noted that bulk ablation and/or reduction in tissue volume potentially occurs over the course of hours, days, and/or weeks after electrical field exposure of the tissue. For example, cells which are electroporated potentially undergo traumatic and/or apoptotic cell death which leads to clearance of their remains and reduction of volume in tissues of the heart.

FIG. 1C shows a cross section of an HCM heart. FIG. 1D shows massive hypertrophy visualized in an echocardiogram (Braunwald's Heart Disease, 9th Edition, Chapter 69).

### Balloon-Positioned Embodiments

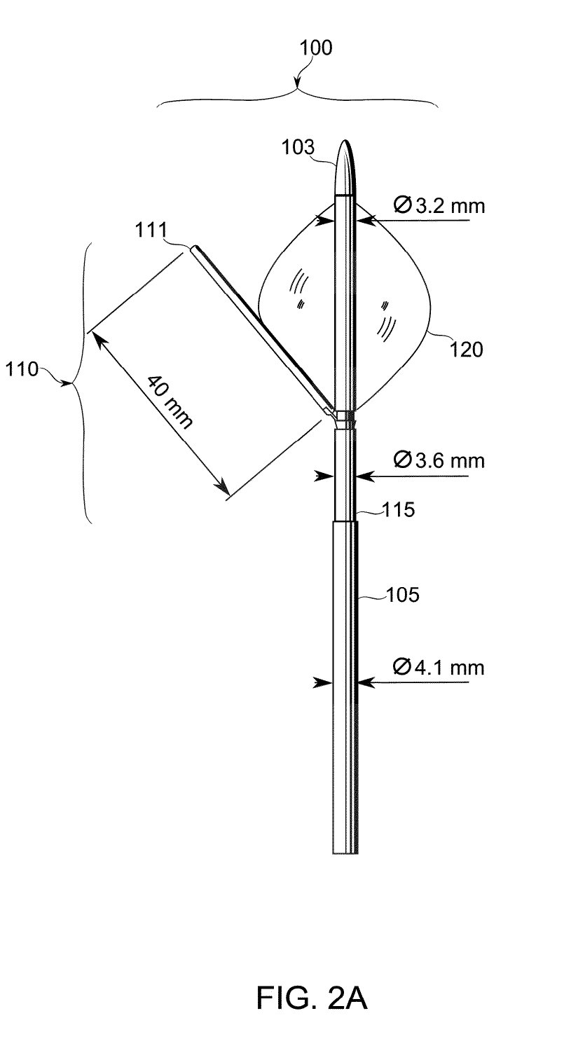

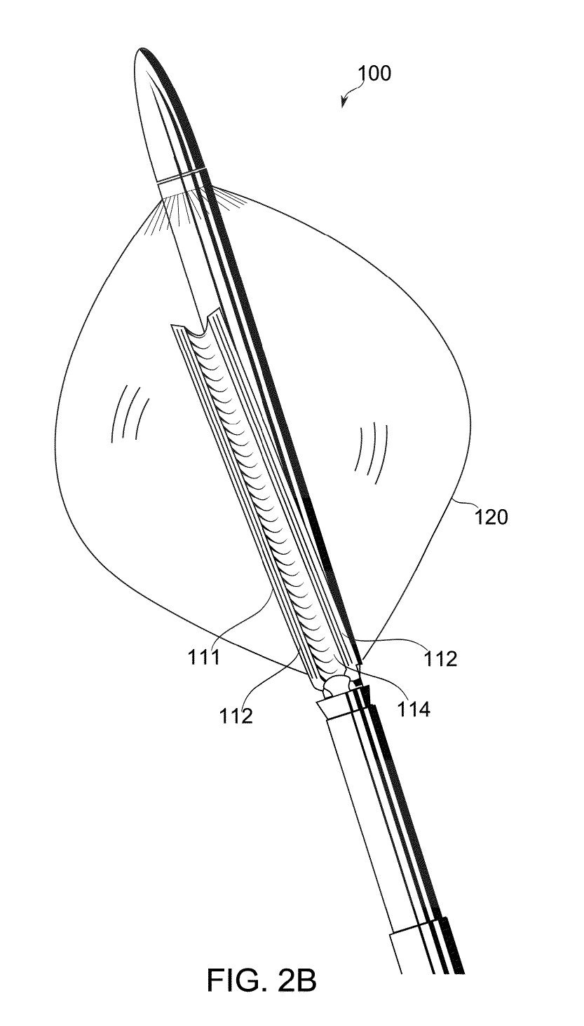

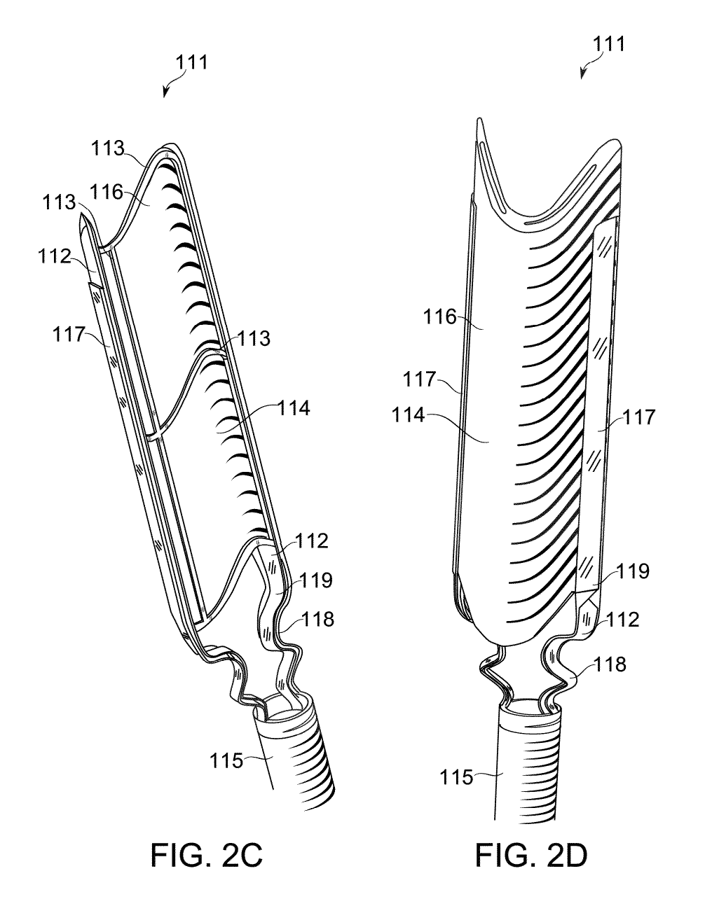

Reference is now made to FIGS. 2A-2B, which show views of an electrode catheter provided with a balloon for pressing the electrode into position for electroporating ablation of myocardial tissue.

In some embodiments of the invention, a transcatheter 100 is used to reach a cavity of the heart for treatment. For example, the left ventricular cavity is reached by inserting transcatheter tip 103 through the aortic valve. In some embodiments, the heart cavity is the right ventricle. In some embodiments, heart chamber access is transapical. In some embodiments, the transcatheter insertion is transfemoral, and occurs, for example, over a guidewire from the femoral artery to the aorta, aortic arch, and across the aortic valve into the heart.

In some embodiments of the invention, once within a ventricular cavity, an electrode assembly 111 carried by the transcatheter 100 is positionable to contact and/or come into proximity with tissue targeted for ablation, for example, the ventricular septum of the left ventricle. In some embodiments, elements of transcatheter 100 are dimensioned for insertion into the heart. For example, sheath diameter is about 3-5 mm. Also for example, electrode length is about 40 mm, 35 mm, 30 mm, or another shorter, longer, or intermediate length.

In some embodiments, positioning comprises withdrawal of a catheter sheath 105 to expose the electrode module 110, including, for example, electrode anchor tube 115 and electrode assembly 111. In some embodiments, positioning comprises inflation of a balloon 120, which presses a hinged electrode assembly 111 away from the body of the microcatheter 100 so that it contacts and/or comes into electrical field proximity with the tissue targeted for ablation. Additionally or alternatively, positioning comprises advancing electrode module 110 distally beyond catheter sheath 105.

Electroporation occurs, for example, according to electrical and temporal parameters as described for electroporation herein. After electroporation, typically lasting a few seconds, the electrode catheter 100 is removed from the heart.

In some embodiments of the invention, transcatheter 100 comprises a balloon 120, for example, a balloon having off-the-shelf availability.

In some embodiments of the invention, frame 111 comprises elongated members 112, between which is arranged electrode surface 114.

Reference is now made to FIGS. 2C-2D, which show views of an electrode assembly 111, used, for example, with the transcatheter of FIGS. 2A-2B.

In some embodiments, electrode surface 114 comprises a flexible printed circuit board 116, along with electrodes 117. Electrodes 117 are, for example, copper coated with gold, for providing high conductivity to the site of treatment. In some embodiments of the invention, the flexible printed circuit board 116 comprises a flexible plastic substrate such as polyimide, PEEK, and/or PET. It is a potential advantage for the electrode surface 114 to be flexible enough to allow expansion of the frame holding the electrodes to its deployed position. In some embodiments of the invention, a stretchable substrate such as a silicone or a polyurethane is used, potentially enhancing conformation to tissue.

In some embodiments, elongated members 112 comprise structural supports 118 (constructed for example from stainless steel and/or Nitinol). Optionally, the elongated members further comprise material such as gold or silver foil 119, for low-resistance conduction of current to the electrodes 117. Electrodes 117, in some embodiments, are provided with current via wires (not shown) which lead through the catheter to an external voltage source. Insulation (for example along portions of elongated member 112) is provided as needed, for example by coating with paralene or another insulating polymer resin. In some embodiments, additional structural support is provided by frame members 113. Optionally, the frame members 113 are configured to adopt a configuration upon deployment that establishes a predefined spacing between electrodes 117. Potentially, predefined spacing helps determine the field strength established between electrodes upon receiving an electroporating electrical potential. Spacing between electrodes is about, for example, 5 mm, 10 mm, 15 mm, 20 mm, or another larger, smaller, or intermediate spacing. In some embodiments of the invention, spacing between electrodes is variable along the extent of the electrodes, within a range of, for example, ±1 mm, ±2 mm, ±3 mm, or another range of spacing which is intermediate, larger, or smaller. In some embodiments of the invention, the tolerance within which an electrode array assumes the electrode spacings of a predetermined deployment configuration is about ±5%, ±10%, ±15%, or another larger, smaller, or intermediate tolerance. It is to be understood that this configuration potentially flexes, for example, due to changing compression from the heart; in some embodiments, the relevant configuration (or configurations) occurs when the electrode array is generating an electroporating electrical field.

Reference is now made to FIGS. 2E-2F, which are schematic views of intercalating electrode pairs 117B-117C and 117D-117E, according to some exemplary embodiments of the invention.

In some embodiments, an electrode 117B-117E comprises a shape such as a comb, spiral, or other shape having extensions. In some embodiments, an electrode shape comprising extensions intercalates with another electrode 117B-117E. A potential advantage of such a configuration is to provide additional control of the shape of the electroporating field extending through a bulk of tissue from the electrodes. Potentially, intercalating electrode projections allows a wider extent of tissue surface to be ablated from, while controlling the depth of ablation to a smaller dimension. Optionally, the maximum field potential difference is decreased in some embodiments comprising intercalated electrodes. In some embodiments, intercalation of electrodes divides a zone of electroporation into 2, 3, 4, 5 or more sub-zones comprising regions within which the pair of closest positive and negative electrode projections is unique. In some embodiments of the invention, boundaries of a single electrode are crossed 2, 4, 6, 8, 10, or more times by a path traversing directly across the whole extent of the electrode array surface.

In some embodiments of the invention, more than one pair of intercalating electrodes is provided. In some embodiments, electrode surfaces are interconnected to themselves and/or to a voltage supply through different layers of a circuit board. Potentially, this allows multiple electrodes (for example, electrodes comprising intercalating extensions) to reach into same the region of electroporation without making mutual contact. It is to be understood that electrodes are separated by sufficient gap and/or insulation such that an applied voltage field does not produce short-circuiting arcing.

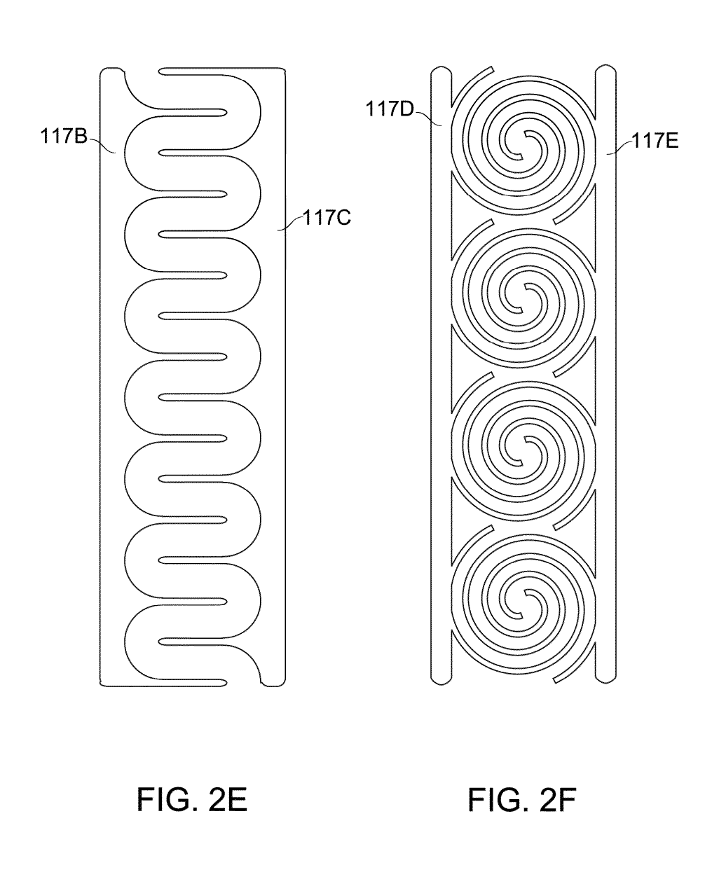

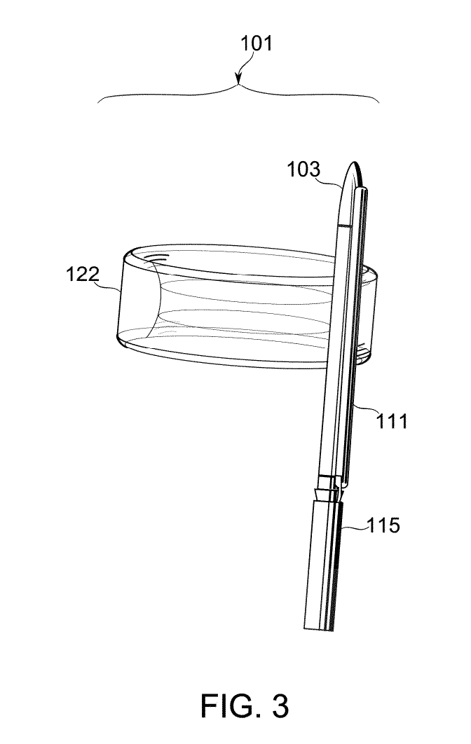

Reference is now made to FIG. 3, which is a schematic view of an electrode catheter in which the electrode array is pressable into position by a balloon 122 shaped to allow the passage of blood therethrough when inflated, according to some exemplary embodiments of the invention.

In some embodiments, blood volume is reserved within a lumen of the balloon 122, such that the pumping of the heart can continue to provide circulation while occupied by the deployed transcatheter. In some embodiments, balloon 122 is inflated to a pressure such that it alternately expands and collapses according to the beating of the heart, while still keeping electrode array 111 pressed against a targeted wall of the heart.

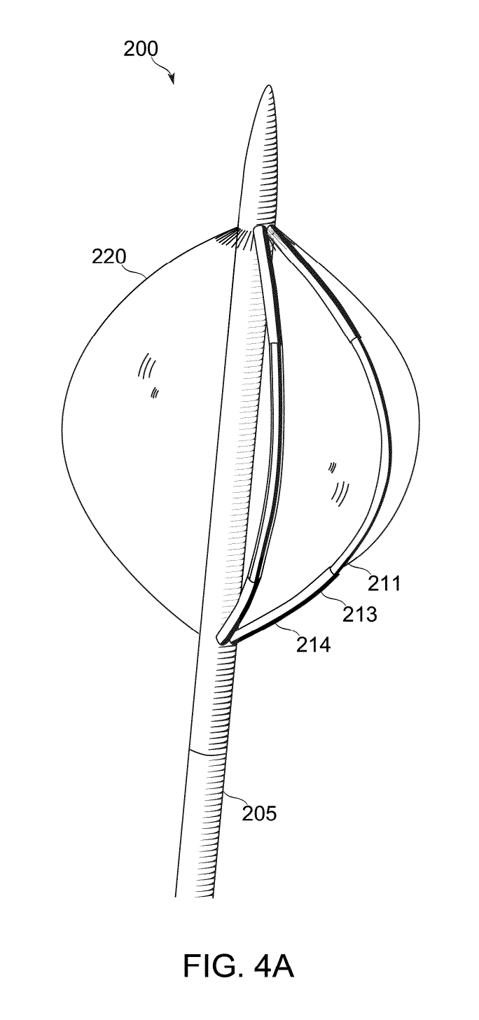

Reference is now made to FIG. 4A, which is a schematic view of a radially expandable electrode array comprising a pair of electrodes 211 pressed into a deployed position for electroporating ablation of myocardial tissue by inflation of a balloon 220, according to some exemplary embodiments of the invention.

In some embodiments of the invention, a pair of electrodes 211 are provided which are carried on deployment arms 213, which connect at either end to transcatheter 205. In the undeployed condition, arms 213 flatten against the body of transcatheter 205. When balloon 220 is inflated, it pushes the arms 213 into a bowed configuration, pressing the electrodes 211 outward. Inserted to a heart, the inflated configuration brings electrodes 211 into contact and/or electrical field proximity with target tissue in preparation for electroporating ablation.

In some embodiments, arms comprise insulating sheaths 214 which can be longer or shorter on either side of the electrode 211 to configure the position at which the electrode potentially contacts and/or comes into closest proximity with heart tissue. In a typical configuration, one of the electrodes is configured to receive a positive voltage, relative to the other arm at a reference voltage. The field is thus defined by the relative positions of the two electrodes upon a single carrier catheter.

Reference is now made to FIG. 4B, which is a schematic view of a radially expandable electrode array comprising multiple electrodes 211A, 211B, 211C, 211D carried on arms 213 and pressed into position for electroporating ablation of myocardial tissue by inflation of a balloon 220, according to some exemplary embodiments of the invention.

In some embodiments, more than two electrodes 211A, 211B, 211C, 211D are provided on the transcatheter's distal end. Optionally, the electroporating electrical field is applied between any two selected electrodes of the electrode array. Optionally, the field is adjusted to reflect the distances between selected electrodes; for example, the voltage differential can be increased for delivery of a pulse between electrodes 211A and 211D relative to an electroporating voltage differential applied between 211B and 211C. In some embodiments of the invention, pulses are delivered alternately to different pairs of electrodes. In some embodiments, the intensity (pulse voltage, frequency, number, and/or duration) of the electroporation protocol is limited by a requirement to avoid of localized heating leading to thermal damage. Potentially, heating is most intense where fields are most strongly concentrated, and in particular, at the electrodes themselves. In some embodiments, however, it is a potential advantage to deliver electroporating pulses within as brief a period as practical, to reduce interruptions to heart function due to the insertion and/or deployment of the transcatheter's electrode array. By switching among electrodes for delivery of electroporating pulses, in some embodiments, electroporation protocol times are reduced, while distributing heating effects over a wider area, and potentially reducing thermal damage effects; in particular, reducing focal thermal damage effects. Thus, for example, electroporation potentials are alternately delivered (optionally with differences in potential corresponding to differences in electrode distance) between electrode pairs 211A, 211C; 211B, 211D; 211A, 211B; and/or 211C, 211D. In such a configuration, each electrode potentially participates in only half of the activated pairings. With appropriate selection of activation pattern, tissue regions located between electrodes 211A and 211D potentially receive approximately equivalent electroporation field exposures, while spreading thermal effects more evenly through time and/or across the tissue. Additionally or alternatively, operation of electrodes can be deliberately manipulated to supply more or less electroporation field strength and/or pulses to different regions of tissue, for example, to manipulate the depth of electroporation across a region targeted for bulk ablation. In some embodiments, the distance of separation between electrodes which are not co-activated is chosen to prevent ohmic heating from rising above the threshold of thermal damage in a region of mutual heating. In some embodiments, the distance from one electrode to the nearest non-co-activated electrode is relatively small: for example, 1 mm, 2 mm, 3 mm, or another smaller, larger, or intermediate distance. It is a potential advantage for non-co-activated electrode pairs to be offset far enough from each other to materially reduce overlapping heating effects, while also being offset minimally enough to electroporate substantially the same region of tissue (having bounds within, for example, 1-3 mm).

While this has been described in relation to a relatively simple electrode layout (four radially spaced electrodes), the concept is applicable in general to any addressable configuration of electrodes in an array. In some embodiments, the number of electrodes provided is 4, 5, 6, 7, 8, or more electrodes. Optionally, two or more electrodes are carried on a single arm, each electrode comprising alternating regions of exposed and insulated surface staggered in position along the arm relative to other electrode surfaces. By alternating which electrode is activated during a sequence of electroporating pulses, the regions of maximal localized heating along the arm are potentially moved from pulse to pulse sufficiently to avoid thermal damage which might otherwise occur. Nevertheless, the electrical field established between the staggered-electrode arm and another electrode arm (optionally also of staggered design) potentially covers roughly similar regions of tissue no matter which electrode is actually activated. Additionally or alternatively, electrodes are actuatable, in some embodiments, to deliver potentials within a range of field potentials. Thus, in some embodiments, for example, a first electrode delivers 1000 V, a second 500 V, and a third 0 V. The three electrodes working together potentially shape the electrical field to cover a volume which is differently shaped than, for example, activating two electrodes at 1000 V and 0 V only. The particularly described numbers, configurations, and voltages delivered by electrodes are exemplary, and it is to be understood that other combinations of voltage, electrode position, and electrode number comprise embodiments of the invention, to the extent available to one skilled in the art and working according to the disclosures herein.

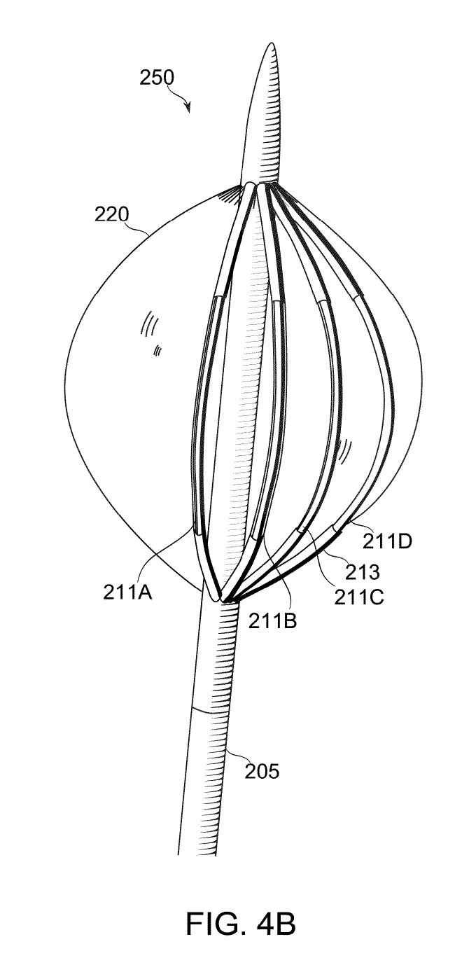

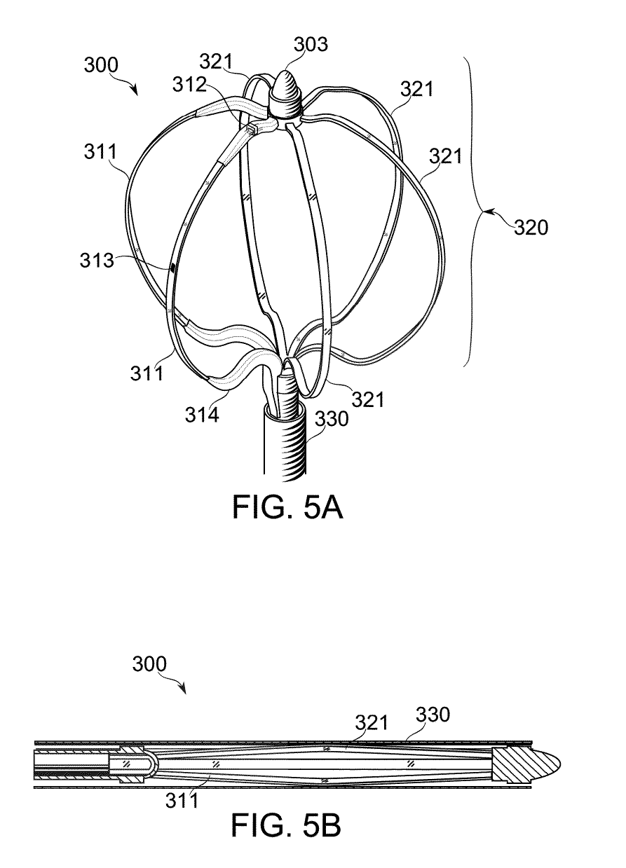

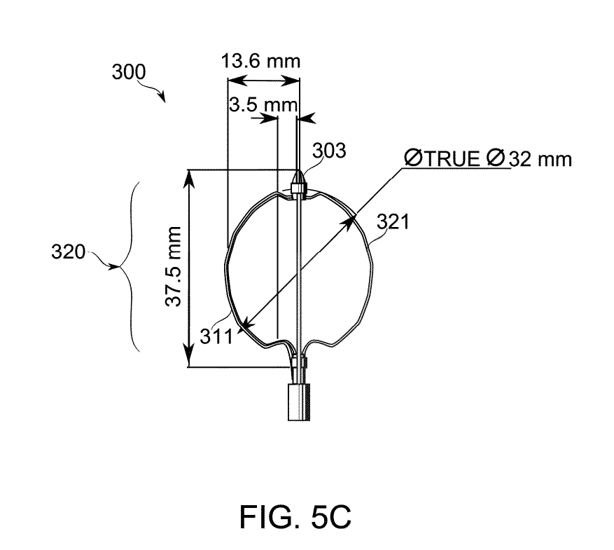

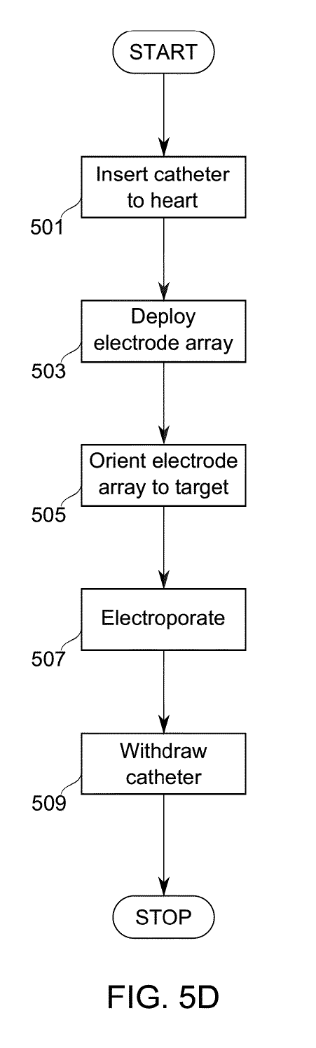

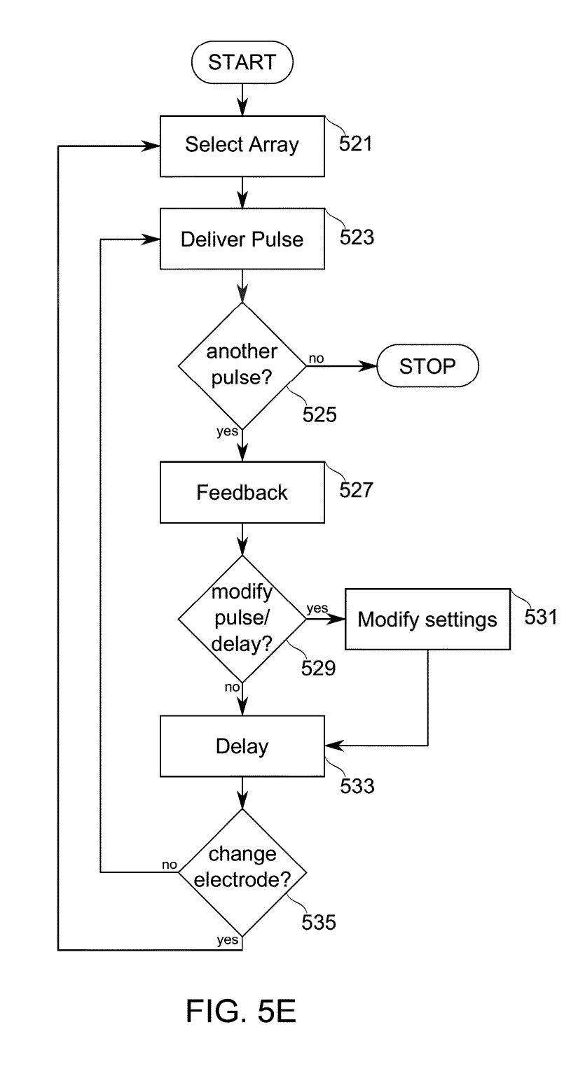

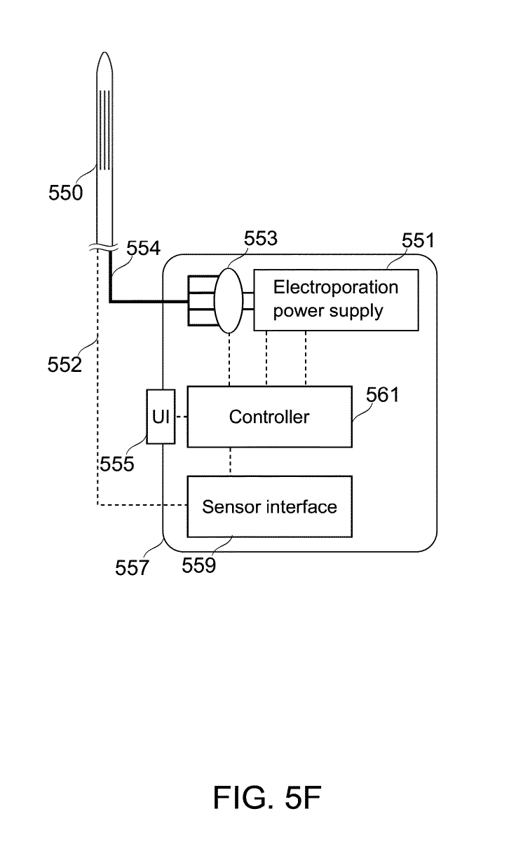

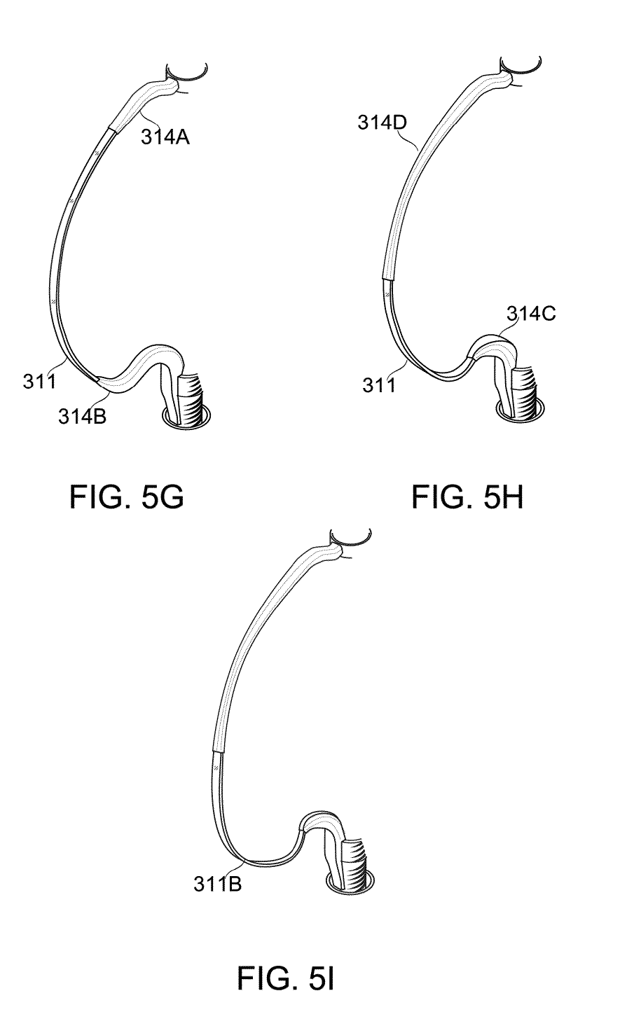

Reference is now made to FIG. 5A, which is a schematic view of a radially expandable electrode array pressed into position for electroporating ablation of myocardial tissue by expansion of a frame, according to some exemplary embodiments of the invention.

In some embodiments of the invention, a basket 320 comprising radially expandable deployment arms 321 is provided at the distal end of a transcatheter 300 for positioning of a plurality of electroporation electrodes 311 within a heart chamber. In some embodiments, electrodes 311 are provided as additions to and/or modifications of the deployable arms 321 on two or more selected arms of the basket. In some embodiments, the number of arms of the basket is, for example, 3, 4, 5, 6, 7, 8 or more arms. In some embodiments, the extent of the electrode 311 is at least partially limited by the complementary extent of insulating layers 314. Optionally, monitoring sensing is provided, for example thermistor 313 provided on one or more of the arms comprising electrodes 311. Optionally, one or more radio-opaque markers 312 are provided to permit fluoroscopic orientation of the device when positioning electrodes.

A potential advantage of a self-deploying basket is a relatively open structure for the passage of blood therethrough. This potentially removes or reduces blockage of blood flow during electrode array deployment, allowing increased time for positioning of the array and/or for electroporating ablation of tissue.

In some embodiments of the invention, tip 303 is inserted into a heart (for example, as shown in FIG. 1B), and sheath 330 withdrawn from the tip, allowing self-expanding of the cage or basket 320. Additionally or alternatively, tip 303 is advanced forward from sheath 330 to allow self-expanding.

In some embodiments, arms 321 of basket 320 are comprised of a shape-memory metal, for example, Nitinol. Arms 321 not electrically involved in electroporation are kept electrically isolated from the electroporation circuit. Optionally, the non-conducting arms are coated with an electrical insulator.

In some embodiments of the invention, positioning of electrodes comprises attachment of the electrodes themselves to the heart wall. For example, electrodes are deployable separately and/or in combination, in some embodiments, to penetrate the heart wall. Optionally, an electrode catheter is used to position and insert the electrodes. In some embodiments of the invention, electroporation parameters are determined based on an observed (for example, fluoroscopically observed) actual distance of electrode insertion. In some embodiments of the invention, electroporation parameters are determined based on the observed conductance between the electrodes, or another electrical property of the circuit created by electrode insertion. A potential advantage of this mode of operation is to allow greater flexibility of electroporated region determination.

Reference is now made to FIG. 5B, which is a cross-sectional view of the electrode array and frame 320 of FIG. 5A, held in a collapsed configuration by a sheath 330, according to some exemplary embodiments of the invention.

In some embodiments of the invention, the electroporation basket 320 is introduced to the region of the heart in a collapsed configuration. Withdrawal of sheath 330 proximally relative to the position of the basket 320 (comprising electrodes 311 and arms 321) allows expansion to the form of FIG. 5B.

Reference is now made to FIG. 5C, which is a schematic view of an array of electrode 311 with an expandable frame 320, with exemplary dimensions thereof, according to some exemplary embodiments of the invention.

The view of some arms 321 of the expandable array is suppressed to illustrate dimensions.

In some embodiments, length of the basket 320, from tip 303 to basal insertion point of the arms 321, is about 37.5 mm. In some embodiments, the length is, for example, 30 mm, 35 mm, 40 mm, or another intermediate, larger, or smaller length. In some embodiments of the invention, the lateral extent of the fully deployed basket is about 13.6 mm in radius. In some embodiments, the lateral extent of the fully deployed basket comprises a radius of about 12 mm, 13.5 mm, 14 mm, 15 mm, 16 mm, or another intermediate, larger, or smaller radius. In some embodiments, the basket 320 itself, when fully deployed, is sized to fit within a sphere of diameter of about, for example, 32 mm. In some embodiments, the basket diameter is about 26 mm, 28 mm, 30 mm, 32 mm, 34 mm, or another intermediate, larger, or smaller diameter. In some embodiments of the invention, a core region (comprising, for example, portions of the arms 321 which are indented from the outer radius) has a radius of about 3.5 mm when the basket 320 is fully deployed. The size of the core radius, in some embodiments, is about 3 mm, 3.5 mm, 4 mm, or another intermediate, larger, or smaller radius.

Reference is now made to FIG. 5D, which is a flowchart of operations for electroporating ablation in a heart, according to some exemplary embodiments of the invention.

At block 501, in some embodiments, the flowchart begins, and an electrode transcatheter for electroporation is inserted to the heart.

At block 503, in some embodiments, the electrode array is deployed. In some embodiments, deployment comprises removal of an electrode array assembly from a protecting and/or restraining sheath. In some embodiments, deployment comprises inflation of a balloon, the balloon being configured to press electrodes outward to contact and/or achieve a predefined proximity to a bulk of the heart wall. In some embodiments, deployment comprises self-expansion of a basket assembly, the arms of the basket assembly being configured to press electrodes outward to contact and/or achieve a predefined proximity to a heart wall.

At block 505, in some embodiments, the electrode array is oriented to the ablation target. Orientation to the ablation target, in some embodiments, comprises turning the electrode array such that selected electrodes are in closest proximity to the target region; optionally, in contact with the target region. In some embodiments, orientation is performed under fluoroscopic observation, assisted, for example, by one or more radio opaque markers. In some embodiments, orientation to the ablation target comprises pressing and/or pulling the electrode array by manipulating the electrode catheter so that it contacts a selected portion of the cardiac wall, for example, the cardiac wall adjacent to the valves of the ventricle, or an apical region of the ventricle.

At block 507, in some embodiments of the invention, electroporation is performed. In some embodiments of the invention, parameters of electroporation are as described hereinabove with respect, for example, to pulse duration, field strength, applied voltage differential, number of pulses, time over which pulses are delivered, and/or frequency of pulses. A typical electroporation protocol, to give a specific example chosen from those described hereinabove, comprises about 40 pulses delivered over about 10 seconds at about 4 Hz, each pulse being about 100 μsec in duration. The field strength in such a typical electroporation protocol is, for example, about 1000 V/cm, corresponding to an applied differential of about 2000 V for an electrode separation of 20 mm.

At block 509, in some embodiments, the electrode transcatheter is withdrawn from the heart, and the flow chart ends.

In some embodiments of the invention, electroporation comprises more than one sequence of the actions described in connection with blocks 501-509 hereinabove. For example, the electroporation procedure is optionally repeated within the same catheterization procedure (with or without withdrawal of the electrode transcatheter from the heart as in block 509). Additionally or alternatively, the electroporation procedure is optionally repeated in a second and/or subsequent catheterization procedure for example after a week or more. It is a potential advantage to remove thin tissue sections sequentially, rather than a single block all at once, for example, to avoid overly weakening the heart by taking too much at one time.

Reference is now made to FIG. 5E, which is a flowchart of operations during an electroporating pulse delivery to a heart, according to some exemplary embodiments of the invention.

At block 521, in some embodiments of the invention, the flowchart begins, and electrodes of the array are selected for activation by an electroporating field pulse. For embodiments of the invention in which the electrode array comprises two electrodes, both electrodes are selected. In some embodiments of the invention, two electrodes selected from among a larger set of electrodes are selected. In some embodiments, three or more electrodes are selected.

In general, at least one electrode is selected as the ground electrode, and at least one electrode is selected through which the field is applied. In some embodiments of the invention, one or more intermediate field electrodes are selected, potentially allowing the field to be shaped between two electrodes by a potential applied to an at least third electrode.

At block 523, in some embodiments, an electrical pulse is delivered. In some embodiments, an individual pulse is for example, 100 μsec long, 150 μsec long, or another pulse duration as described herein. In some embodiments, an applied voltage differential is about 1000 V, or another applied voltage differential as described herein. In some embodiments, applied voltage varies over time. For example, an initially higher voltage is applied to initiate electroporation, with a lower voltage used during later stage of the pulse, such that heating effects are reduced. Current delivered is, for example about 1 Amp.

At block 525, in some embodiments, a determination is made to deliver another pulse—or not. If not, the flowchart ends. If yes, the flowchart continues. The determination to deliver another pulse, in some embodiments, is based, for example, on an elapsed count of pulses delivered in the current sequence. Additionally or alternatively, in some embodiments, pulse delivery is terminated when a temperature or other safety threshold is exceeded. In some embodiments, pulses are delivered as opportunity permits (for example, while there is good contact and/or sufficient proximity during a particular heartbeat phase) during a predetermined period. These examples of pulse-continuation determination criteria are exemplary, and not limiting.

At block 527, in some embodiments, sensor feedback is optionally evaluated in anticipation of modifications to the timing and/or intensity of the next electroporation pulse to be delivered. For example, feedback from one or more temperature sensors is used to determine if an additional pause should be inserted to allow cooling before a next pulse is delivered, and/or to determine if a pulse voltage and/or duration should be reduced to prevent overheating. In some embodiments of the invention, changes in tissue resistance over time during pulses are monitored to help determine if pulse parameters are efficacious in inducing electroporation. Optionally, pulse duration and/or voltage is adjusted based on observed and/or targeted changes due to electroporation in the target tissue. In some embodiments of the invention, feedback comprises imaging of the heart, for example of heart function, for determining that a volume of the heart has been inactivated, potentially reflecting the bulk which has been irreversibly electroporated, leading to tissue ablation.

At block 529, in some embodiments, a determination is made to modify pulse and/or pulse delay settings. If yes, the pulse settings are modified at 531, for example as described in connection with feedback considerations mentioned for block 527, and the flowchart continues at block 533. If no, the flowchart continues at block 533.

At block 533, in some embodiments of the invention, a delay is introduced. The delay, in some embodiments, is simply the delay required to obtain a preset frequency of electroporation pulses, for example, 1 Hz, 2 Hz, 4 Hz, or another larger, smaller, or intermediate frequency of electroporation pulses. In some embodiments, the delay includes synchronization delays to assure a particular heartbeat phase during pulse delivery. In some embodiments, the delay is optionally gated by sensor feedback, for example, by determination that the electrode is in a desired state of electrical contact and/or within a desired temperature range.

At block 535, in some embodiments, a determination is made whether or not the selected electrodes should be changed. In embodiments where multiple electrodes are available for selection, the determination is, for example, according to a preset pattern, according to feedback indicating which electrodes are ready for the next electroporation pulse, and/or another criterion. If no reselection of the electrodes to be activated is required, the flowchart continues with block 532. If reselection is required, the electrodes are again selected at block 521, and the flowchart continues.

Reference is now made to FIG. 5F, which schematically illustrates a system for electroporating ablation of heart tissue, according to some exemplary embodiments of the invention.

In some embodiments of the invention, a system for electroporating ablation of heart tissue comprises an electrode catheter 550, constructed, for example, according to the descriptions of FIGS. 2A-5C, and/or 5G-5I, connected to an electroporating base station 557. In some embodiments, electroporating pulse voltages generated by electroporation power supply 551 are transmitted to the electrode catheter 550 over pulse voltage lines 554. Optionally, a selector unit 553 selects electrodes over which pulses are delivered. In some embodiments, the power supply connections to the electrodes are fixed. In some embodiments, the electrode power supply is suitable to deliver pulses of the voltage, duration, and/or frequency required to create electroporating fields in the heart, for example as described herein. In some embodiments of the invention, a controller 561 is provided which controls electroporation parameters such as voltage, electrode selection, and/or timing. In some embodiments, a user interface 555 is used to activate the electroporation protocol and/or select parameters thereof. In some embodiments, a sensor interface 559 is provided, which detects parameters, for example, temperature and resistance, related to operation of the electroporating array. In some embodiments, another parameter is sensed, for example, heartbeat phase. Optionally, controller 561 uses sensor information in determining pulse control parameters.

Reference is now made to FIGS. 5G-5I, which schematically illustrate variations on the shape and exposed regions of an electroporating electrode for use within a lumen of the heart, according to some exemplary embodiments of the invention.

In some embodiments, the extent of electrical insulation (for example, insulating “socks” 314A-314D, or another insulating structure), at least partially determines the region of heart muscle which will receive electroporating pulses from the electroporation electrodes 311. Additionally or alternatively, the extent of the electrode itself is adjusted for this determination. For example, in FIG. 5G, both insulating members 314A and 314B are relatively short, exposing a long intermediate section of electrode 311 for potential contact (physical and/or electrical) with heart muscle. In FIG. 5H, on the other hand, one of the insulating regions 314D is extended to a larger portion of the support member, such that electrical contact is made only near the proximal end of the basket. It is a potential advantage to allow adjusting of a similar electrode shape to better match the requirement for tissue ablation in a particular subject by extending and/or reducing the exposed electrode region.

In some embodiments, the shape of the electrode itself is varied. For example, the electrode 311B of FIG. 5I comprises a flattened region near the proximal end of the electrode basket, compared, for example, to the electrode of FIG. 5H. Potentially, this results in more complete contact with and/or closer electrical field proximity to tissue adjacent to the core of the device. In embodiments where the transcatheter is designed to be inserted through a heart valve, this tissue is potentially the tissue most likely to interfere with valve operation. It is a potential advantage for treatment of hypertrophic heart muscle tissue to specifically target reduction in the pressure gradient which hypertrophic tissue generates in the vicinity of the valves of the heart. Such a pressure gradient, when high, reflects resistance to flow. Thus, electrodes and/or their support members are advantageously designed, in some embodiments, to press against the heart tissue to the sides of and/or surrounding the valves of the left ventricle.

### EXAMPLES

Endovascular Smooth Muscle Ablation in Rodents

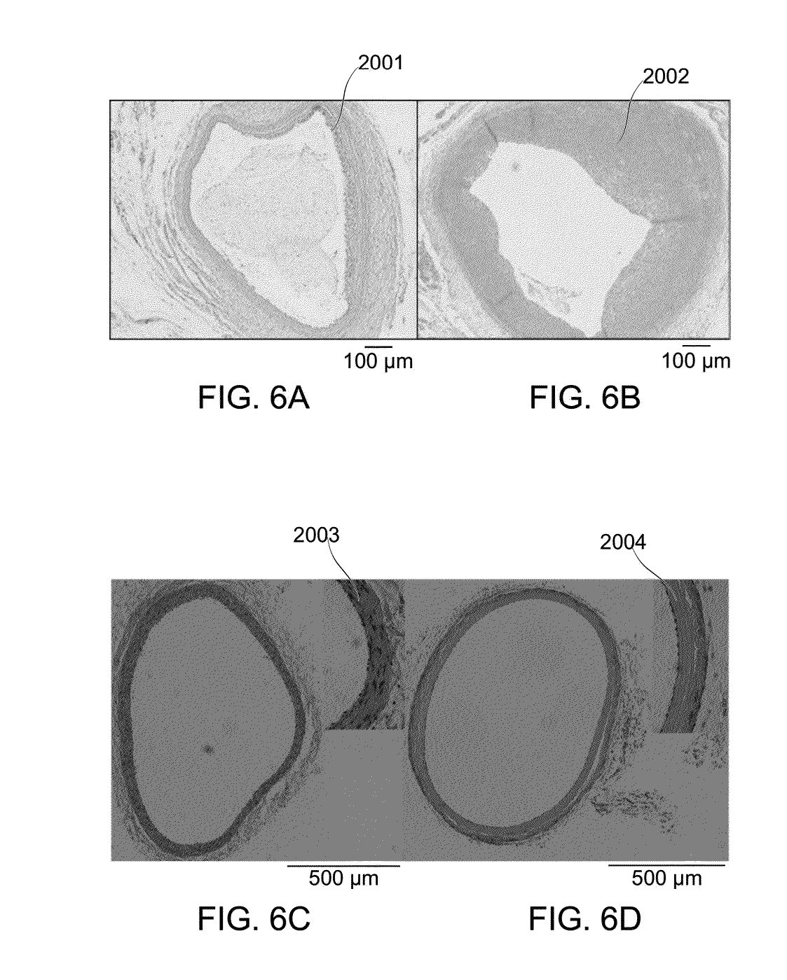

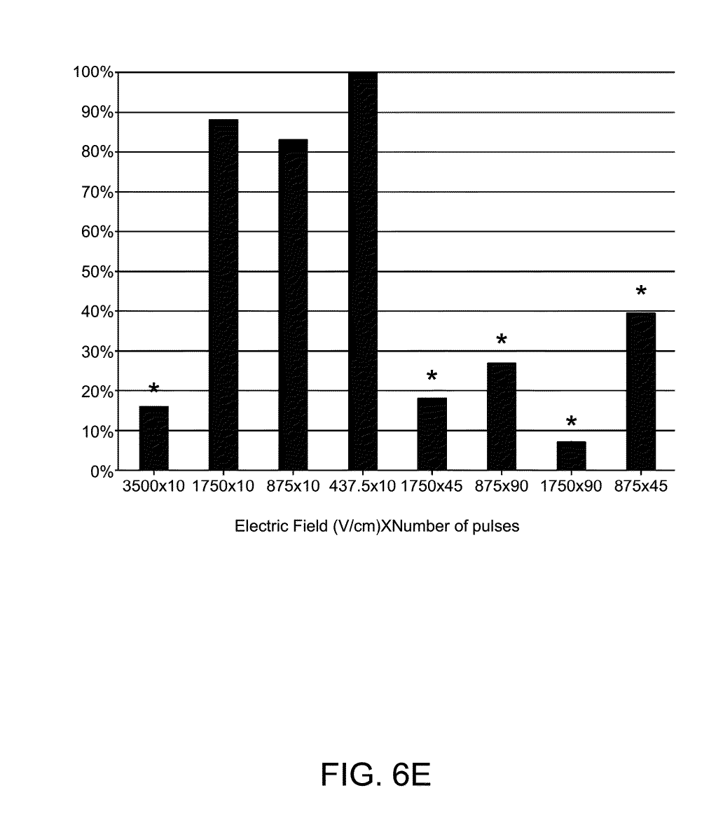

Reference is now made to FIGS. 6A-6E, which describe results of NTIRE protocols on vascular smooth muscle cells (VSMC) of rodent carotid artery under a range of electroporation conditions, reflecting general cardiovascular effects of NTIRE, according to some exemplary embodiments of the invention.

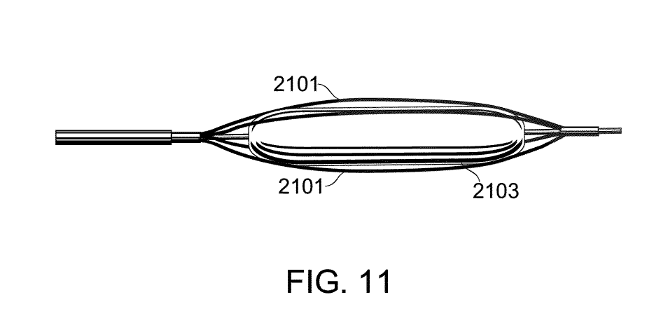

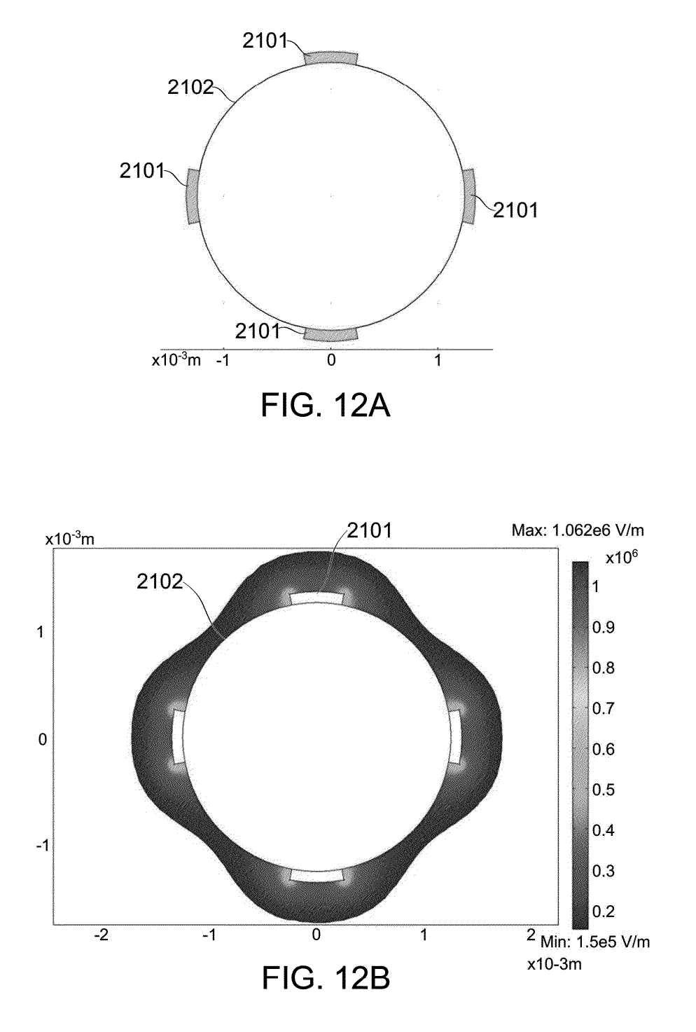

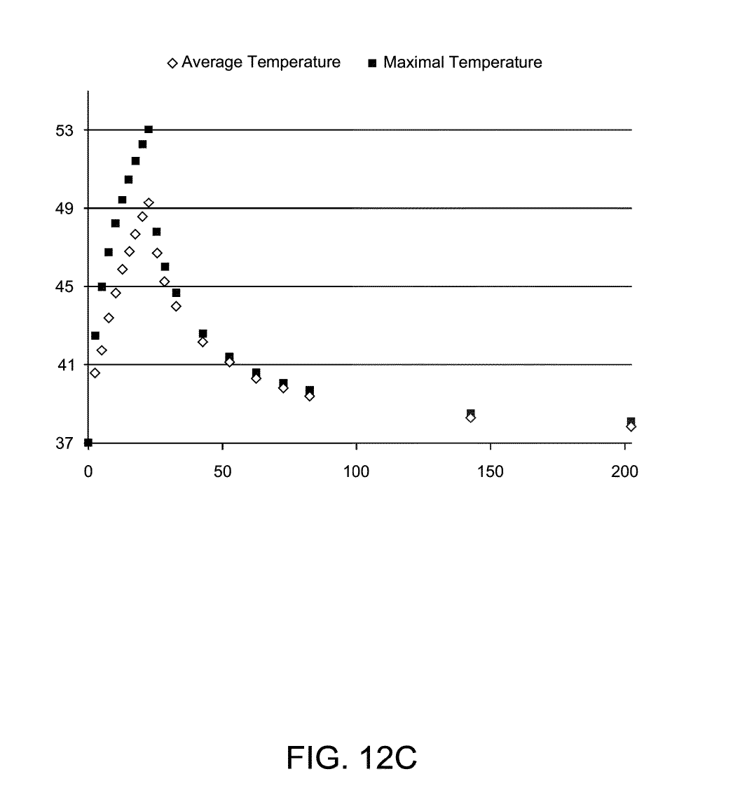

FIG. 6E is a graph showing the effects of different electroporation parameters on VSMC in rodent carotid artery (N=5 for group 3500×10, and N=4 for all other groups). The bars show ablation effect as the percentage of VSMC cells remaining in a treated left carotid artery compared with the right carotid artery of the same animal 7 days post-treatment. The reduction in five of the groups was statistically significant (P<0.001, bars marked with an asterisk). It can be seen that the same pulse parameters are potentially more or less effective, depending strongly on the number of repetitions provided. Furthermore, a range of voltage field strengths are potentially effective, but the number of pulses required for effective electroporating ablation is dependent on field strength. It will be seen moreover (for example, in connection with modeling described hereinbelow in relation to FIGS. 11 and 12A-12C, and Table 2) that heating effects (and therefore, thermal damage) are also dependent on these parameters. Finally, it is a potential advantage to reduce the total period over which pulses are provided, to reduce the period during which circulation through the heart is mechanically and/or electrically interfered with. By choosing parameters from within the parameter space which are associated both with effective electroporation of cellular components of cardiovascular muscle, and with substantial avoidance of thermal damage, an electroporation protocol can be determined which is potentially useful for myocardial NTIRE.

FIGS. 6C-6D are micrographs demonstrating complete ablation of a VSMC population one week following NTIRE with 90 pulses of 1,750 V/cm (FIG. 6D), compared with right carotid artery of the same animal that was used as a control (FIG. 6C). The complete absence of VSMC cells 2003 present in the control can be seen in the treated vessel, accompanied by notable repopulation of the endothelial layer with endothelial cells 2004.

FIGS. 6A-6B are micrographs demonstrating intimal denudation 2001 of rodent carotid artery at 28 days after treatment, compared to a control untreated region 2002.

Endovascular Smooth Muscle Ablation in Large Animals

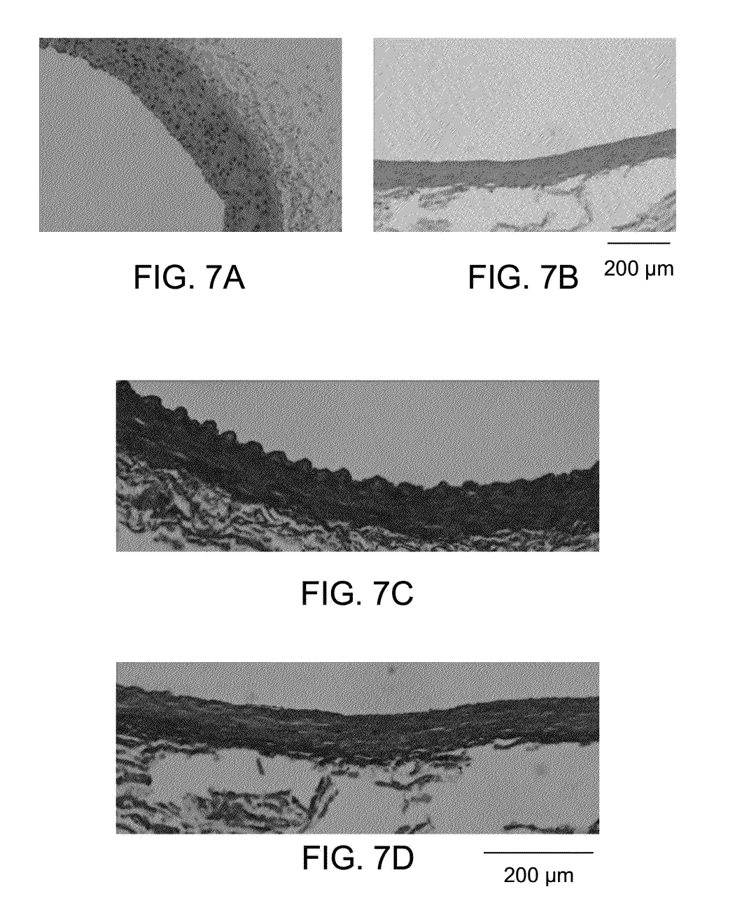

Reference is now made to FIGS. 7A-7D, which are photomicrographs showing decellularization by NTIRE of blood vessels in rabbit, illustrating general characteristics of NTIRE decellularization in cardiovascular tissue, according to some exemplary embodiments of the present invention;

An endovascular device with four electrodes on top of an inflatable balloon applied electroporation pulses. Right iliac arteries of eight rabbits were treated with 90 NTIRE pulses. Angiograms were performed before and after the procedures. Arterial specimens were harvested at 7 and 35 days. Evaluation included hematoxylin and eosin (H&E), elastic Von Giessen, and Masson's trichrome stains. Immunohistochemistry of selected slides included smooth muscle actin, proliferating cell nuclear antigen, von Willebrand factor and S-100 antigen. At 7 days, all NTIRE-treated arterial segments displayed complete, transmural ablation of vascular smooth muscle cells (FIGS. 7A-7B). FIG. 7A shows a control iliac artery, while FIG. 7B shows electroporation-treated iliac artery. The thickness and cellularity of the tunica media is relatively decreased in FIG. 7B.

At 35 days, similar damage to VSMC was noted. In most cases, elastic lamina remained intact and endothelial layer regenerated (FIGS. 7C-7D). FIG. 7C shows a control iliac artery, while FIG. 7D shows electroporation-treated iliac artery. Cellularity (reddish color) is reduced, while collagen fibrils (purple) remain intact in the treated vs. untreated conditions. Occasional mural inflammation and cartilaginous metaplasia were noted. After five weeks there was no evidence of significant VSMC proliferation, with the dominant process being wall fibrosis with regenerated endothelium.

Results demonstrate that NTIRE can be applied in an endovascular approach. Results demonstrate that NTIRE efficiently ablates vessel wall (cardiovascular muscle) within seconds, with no damage to extra-cellular structures.

NTIRE Ablation of the Beating Heart

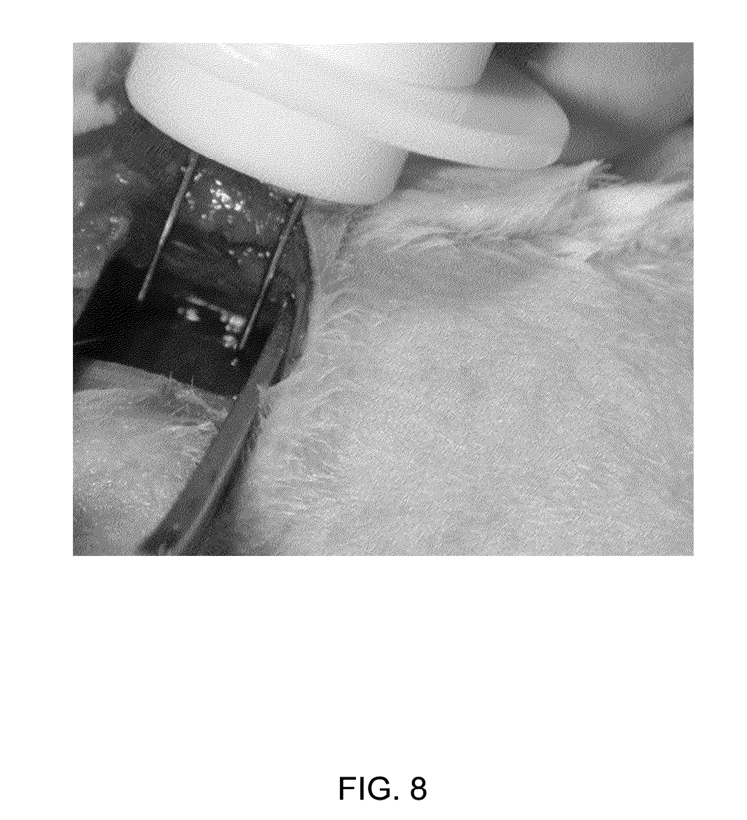

Reference is now made to FIGS. 8, 9A-9F, and 10A-10B, which describe methods and results of NTIRE ablation in a mammalian heart, according to some embodiments of the present invention.

Methods:

A study was performed at the Neufled cardiac research center, Sheba Medical Center. Animal experiments were approved by the local ethical committee (animal protocol number 715/12).

Sprague Dawley (SD) rats were used in the study. Under general anesthesia and endotracheal intubation, sterile thoracotomy was performed. Two needle electrodes were introduced into the anterior wall of the myocardium. FIG. 8 shows needle electrodes during insertion, according to some embodiments of the present invention. In the image, in vivo IRE is achieved using two needle electrodes with a separation of 0.5 cm inserted into the anterior myocardium of an SD rat through the 4th inter-costal space.

Irreversible electroporation was performed by applying 10 direct current electric pulses of 100 microseconds in length at a frequency of 1 Hz. Pulses were applied between two needle electrodes using a high voltage pulse generator intended for electroporation procedures (BTX ECM 830, Harvard Apparatus, Holliston, Mass.). The choice of parameters for inducing irreversible electroporation without thermal damage was performed using COMSOL Multiphysics 4.2 together with MATLAB 2011b to simulate the electric field and heat generated around the needle electrodes. Immediately following the procedure, echocardiography was used to evaluate myocardial damage. One week following the surgical procedure, the efficiency of IRE ablation was evaluated using morphometric analysis of H&E slides.

In an additional experiment, different electroporation protocols were compared to the myocardial damage of infarction due to the occlusion of the anterior descending coronary artery. Voltage differences of 50 V, 250 V and 500 V were applied, and the damage of myocardial infarction compared. All animals underwent echocardiographic evaluation at baseline, after one week and after one month.

Results:

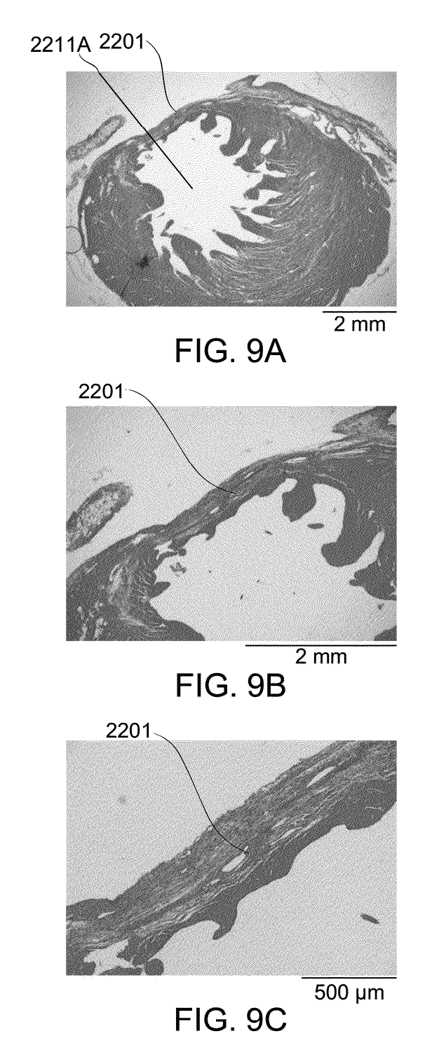

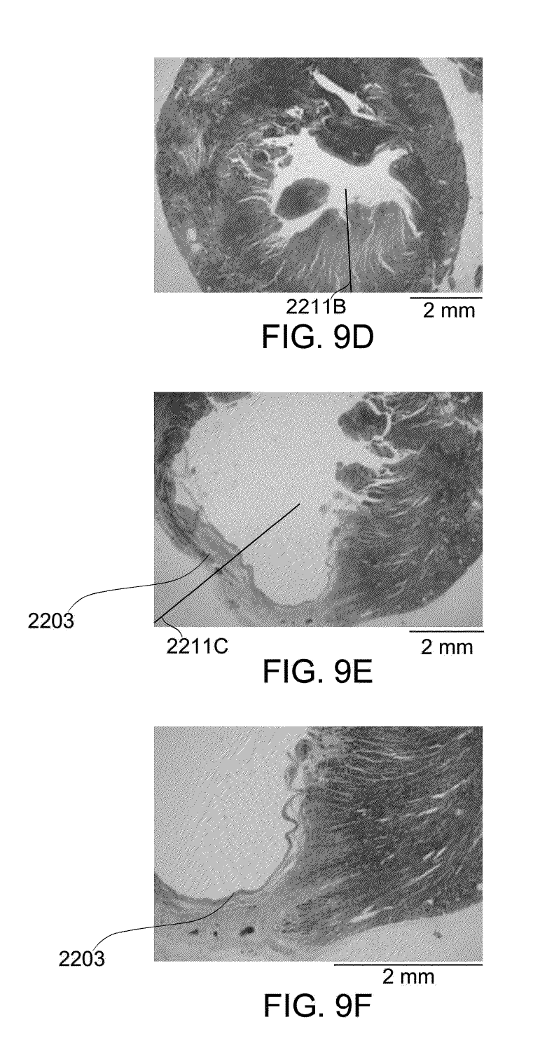

Computer simulation showed that 10 direct currents of 100 microsecond pulses at a frequency of 1 Hz do not induce a significant increase in temperature. In vivo IRE induced myocardial cell death within seconds, without significant arrhythmias or heart failure. Histologic analysis showed that an IRE protocol of 500 V induced a 60% reduction in myocardial thickness at day 7. FIGS. 9A-9C show ablated region 2201 at different magnifications at day 7 post-treatment. The track of electrode penetration is indicated by line 2211A. Penetration occurred parallel to the plane of the histological sections shown, about 2.5 mm to either side (2.5 mm “in” and “out” of the plane of the drawing). Surrounding musculature in the section provides the control. Histologic analysis at 28 days showed that the percentage of scarred myocardium was 36%±18%, 11%±10%, 8%±6% and 3%±4% in the MI, 500 V, 250 V and 50 V groups, respectively. Compared with MI, electroporation scar tissue in the 500 V group was significantly thicker (35% vs. 18% of the normal ventricular wall thickness, FIGS. 9E-9F). FIGS. 9D-9F show Masson trichrome stain of the left ventricle at day 28. FIG. 9D shows a normal control ventricle. FIGS. 9E-9F show the effect of IRE in region 2203 at different magnifications. In FIG. 9D, line 2211B indicates a direction of along which electrode penetration would have occurred had the heart experienced electroporation similar to that experienced along track 2211C of FIG. 9E. Again, penetration is about 2.5 mm in and out of the plane of the drawing.

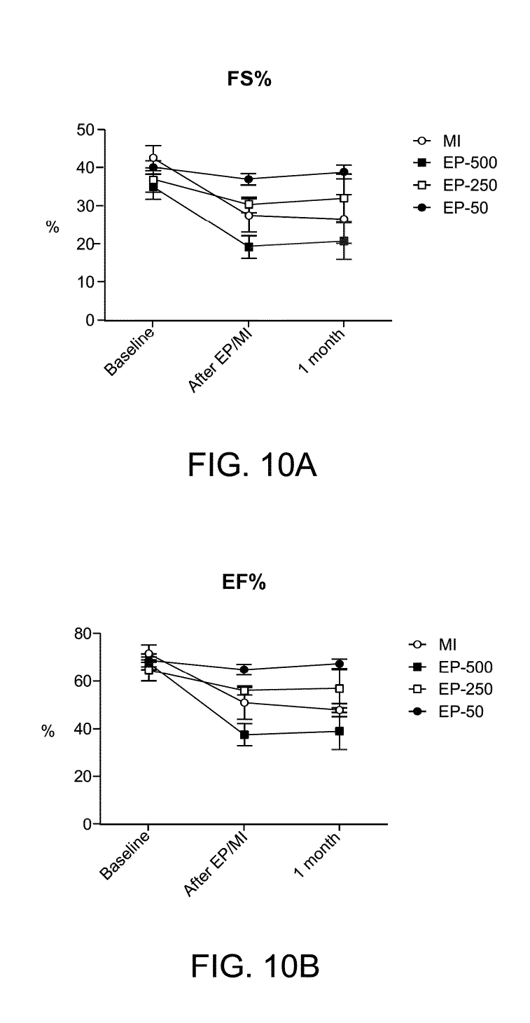

Echocardiographic analysis at 7 and 28 days showed significant deterioration of ejection fraction and fractional shortening in the 500 V and MI groups compared with 250 V and 50 V groups (FIGS. 10A, 10B). The amount of damage in the 500 V group was similar to that of MI.

In FIG. 10A, FS % is fractional shortening; in FIG. 10B, EF % is ejection fraction, as determined by echocardiographic results. Error bars correspond to P values for differences between groups at 0.02 and 0.01 respectively.

Significance:

The study describes an IRE protocol that selectively ablates cellular components in the beating heart with no thermal damage. 10 short electroporation pulses at a frequency of 1 Hz (total treatment duration of 10 seconds) can induce significant transmural damage to the beating heart. IRE damage to the myocardium is shown to be different from the damaged caused by myocardial ischemia following infarction. IRE causes significantly thicker scar tissue without damage to collagen or elastic fibers. Differences between myocardial ischemia and IRE are potentially attributable to the non-thermal nature of IRE and to the fact that this biophysical modality does not damage extra-cellular components.

Theoretical Analysis of NTIRE Parameters

The choice of physical parameters affecting thermal properties and field strength is made, in some embodiments of the invention, according to numerically simulated theoretical considerations, based on the configuration of electrodes provided in an embodiment of the invention.

Reference is now made to Table 1, which gives exemplary values of electrical, thermal, and biological parameters used in theoretical analysis of electrical field production and its effect on tissue heating.

TABLE 1

| QUANTITY | SYMBOL | UNITS | VALUE |
|---|---|---|---|
| Tissue electrical | σ | S · m⁻¹ | 0.6 |
| conductivity | | | |
| Tissue thermal conductivity | K | W · m⁻¹ · K⁻¹ | 0.5 |
| Tissue heat capacity | Cₚ | J · Kg⁻¹ · K⁻¹ | 3750 |
| Tissue density | ρ | Kg · m⁻³ | 1000 |
| Blood heat capacity | C_{b} | J · Kg⁻¹ · K⁻¹ | 3640 |
| Blood density | 1080 | Kg · m⁻³ | 1000 |
| Blood perfusion rate | ω_{b} | | 0.0001 |
| Electroporation pulse | | msec | 0.1 |
| duration | | | |
| Molecular collision | A | sec⁻¹ | 5.6 × 10⁶³ |
| frequency | | | |
| Energy Barrier | E | J · mole⁻¹ | 4.3 × 10⁵ |
| Electrode conductivity | σₑ | S · m⁻¹ | 4.032 × 10⁶ |
| Electrode thermal | Kₑ | W · m⁻¹ · K⁻¹ | 100 |
| conductivity | | | |
| Initial temperature | T₀ | K | 310.15 |
| Gas Constant | R | J · mole⁻¹ · K⁻¹ | 8.13 |

The modeled situation can be considered, in some embodiments, as that of a single pulse delivered in an electroporating electrode configuration surrounded by an infinitely extending biological homogenous isotropic tissue. The model can be in 2 or 3 dimensions. Here, the model configuration evaluated is a radially symmetric array in an endovascular configuration, but the method is readily extended to modeling of electrodes within a heart by one skilled in the art, working based on the descriptions herein.

Joule heating is evaluated, in some embodiments, by solving the Laplace equation for potential distribution:

∇·(σ∇Φ)=0 Equation 1

p=σ|∇Φ|² Equation 2

where Φ is the electric potential (Volts), σ is the electrical conductivity (S/m) and p is the heat generation rate per unit volume (W/m³). The heating of the tissue resulting from electroporation can be calculated by adding the Joule heating source term to the Pennes bio-heat transfer equation:

$$∇ ( k ∇ T ) + {ω}_{b} {c}_{b} ( {T}_{a} - T ) + q + σ {\vert ∇ Φ \vert}^{2} = δ ρ {c}_{p} \frac{δ T}{δ t} \qquad Equation 3$$

where k is the thermal conductivity of the tissue (W·m⁻¹K⁻¹), T is the absolute temperature, ω_{b} is the blood perfusion rate (s⁻¹), c_{b} is the heat capacity of the blood (J·Kg⁻¹K⁻¹), Tₐ is the arterial temperature, q is the basal metabolic heat generation (W/m³), ρ is the tissue density (Kg/m³) and cₚ is the heat capacity of the tissue (J·Kg⁻¹K⁻¹).

Using the transient temperature solution of equation, a kinetic model of thermal damage base on the Arrhenius formulation can be used. The model calculates the Henriques and Moritz thermal damage integral:

$$Ω ( t ) = \int A {e}^{- \frac{E}{RT}} d t \qquad Equation 4$$

where T is the transient temperature from Equation 3, Ω is a dimensionless indicator of damage, A is a measurement of molecular collision frequency (s⁻¹), E is an energy barrier that molecules surmount in order to denature (J/mole), R is the gas constant (J·mole⁻¹K⁻¹) and t is the time (s). The values of A and E are based on experiments in different tissue evaluating different kinds of damage. This analysis is based on values of A and E that are appropriate for thermal damage of human arterial tissue; heart tissue thermal damage constants may be assumed to be similar.

Ω can be calculated, for example, for specified location in the domain Ω(x, y, z) for the maximal temperature in the domain Ωₘₐₓ, and/or for the average temperature of any sub-domain Ω_{average}.

Electroporation pulses are optionally modeled as discrete square DC pulses of length t₁ with a pulse frequency f. Thermal damage analysis takes into account both the resistive heating during the pulses, and the time interval with no resistive heating between the pulses. For multiple pulse electroporation protocols, the problem is optionally solved separately for each time interval (either pulse or inter-pulse pause), with the transient solution at the end of the time-interval used as the initial condition for the next time-interval:

$$Ω ( t ) = {\sum }_{i = 0}^{N - 1} ( {\int }_{i τ}^{i τ + {t}_{1}} A {e}^{- \frac{E}{RT}} d t + {\int }_{i τ + {t}_{1}}^{i τ + {t}_{1} + {t}_{2}} A {e}^{- \frac{E}{RT}} d t ) \qquad Equation 5$$

where N is the total number of electroporation pulses, t₁ is the pulse duration interval, t₂ is the time interval between the end of the pulse and the beginning of the next pulse, and τ is the sum of t₁ and t₂. The frequency of the electroporation protocol is defined as:

$$f = \frac{1}{τ} \qquad Equation 6$$

For the purposes of determining boundary conditions, the problem is modeled, in some embodiments, as a tube (artery or heart chamber) inside a large homogenous tissue block, with the endovascular electrodes located on the inner surface of the tube. For the electric potential equation, the electrodes are represented, in some embodiments, by a fixed voltage (Dirichlet) boundary condition, with one electrode having a positive potential and the other one set to zero:

φ₁=V₀ Equation 7

φ₂=0 Equation 8

where V₀ is the potential difference (volts) applied across the electrodes during the electroporation pulses. A zero electric flux (Neumann) boundary condition is applied, in some embodiments of the invention, at all the boundaries of the model not in contact with the electrodes:

$$\frac{δϕ}{δ n} = 0 \qquad Equation 9$$

For the bio-heat equation, initial temperature in the entire domain is set, in some embodiments of the invention, to the physiologic temperature (310.15° K). The boundaries along the tube, in some embodiments, are taken to be adiabatic (Neumann) boundary conditions to predict the maximal temperature rise along the arterial/heart wall:

$$\frac{δ T}{δ n} = 0 \qquad Equation 10$$

The outer surface of the large tissue block is defined, in some embodiments, as a constant physiological temperature (Dirichlet) boundary condition:

T=310.15° K Equation 11

The foregoing equations are used to solve the problem with a particular electrode geometry, for example using COMSOL Multiphysics and MATLAB, to provide a quantitative estimate of the amount of thermal damage to the arterial/heart wall. The estimate is based on the value of Ω.

Reference is now made to FIG. 11, which shows a radially arranged array of electrodes usable for vascular cell ablation by electroporation.

Reference is now also made to FIGS. 12A-12B, which show a theoretical model of a circularly arranged array of four electroporation electrodes, and associated electrical field densities. In the model, two electrodes are assigned electrical potentials of ΦP=0, and two P=V₀, alternating every 90 degrees. Details of electrical and thermal modelling are presented, for example, in [Maor, 2010], which is hereby incorporated herein in its entirety. For a heart geometry, dimensions of a heart chamber, and dimensions of an electrode array, for example, as described herein, are substituted.

FIG. 12A shows an electrode array, similar, for example, to the array of FIG. 11, in cross-section. Each electrode 2101 is arranged around a central lumen 2102, for example as if distributed around an inflated balloon. FIG. 12B shows associated theoretical electrical field densities for an applied voltage differential of 600 V. Potentially electroporating field levels of >1500 V/cm are shown in the colored region.

Reference is now made to FIG. 12C, which shows maximal and average temperature (° C.) as a function of time (seconds) due to 90 electroporation pulses with ΔV=600 volts, at a frequency of 4 Hz. The maximal temperatures reached are 53.1° C. and 49.3° C., respectively, which, for some transient conditions, is sufficiently low to avoid scarring due to thermal damage.

Reference is now made to Table 2, which shows fraction of thermally damaged molecules for each of several irreversible electroporation protocols. Temperatures near the conductors (and particularly at conductor corners) were significantly higher, resulting in a significant change between the thermal damage integral calculated with maximal temperatures vs. average temperatures. Emphasized lines show terminal voltages of each condition series where thermal damage begins to be significant, representing reaching a limit of voltage above which irreversible electroporation ceases to be non-thermally damaging. In some embodiments of the invention, electroporation protocol parameters are chosen such that an average amount of expected thermal tissue damage comprises damage to no more than 0.5-1.0% of molecules, 1.0-2.0% of molecules, 2.0-2.5% of molecules, or another range of expected molecular damage having the same, intermediate, larger and/or smaller bounds. In some embodiments of the invention, electroporation protocol parameters are chosen such that a maximum amount of expected thermal tissue damage in a tissue region or sub-region comprises damage to no more than 20-40% of molecules, 30-50% of molecules, 40-60% of molecules, 50-80% of molecules or another range of expected molecular damage having the same, intermediate, larger and/or smaller bounds. The region of tissue considered with respect to the average and/or maximum thermal damage threshold can be an area of any size within the influence of the electroporating electrical field. It is a potential advantage to use the thermodynamic model described hereinabove to model thermal damage, as this has the potential, being based on consideration of well-known principles of thermodynamics, to well-approximate actual thermal damage. However, it should be understood that this is not the only way to model and/or set a limit of thermal damage. For example, in some embodiments of the invention, selection for avoidance is relative to a maximum expected temperature reached, a maximum of the excursion from physiological temperature integrated over time, or any other measure which relates to a potential to create thermal damage. For example, a maximum temperature threshold, in some embodiments, is 50° C., 55° C., 60° C., 65° C., or another greater, smaller or intermediate temperature. In some embodiments, the time considered in a determining whether a temperature threshold is exceeded is, for example, 10 μsec, 50 μsec, 100 μsec, 200 μsec, or another greater, larger or intermediate period.

TABLE 2

| | | | | DAMAGED | DAMAGED MOLECULES |
|---|---|---|---|---|---|
| SEQUENCE | ΔV | MAX Ω | AVERAGE Ω | MOLECULES (MAX) | (AVERAGE) |
| 90 pulses, 4 Hz | 400 | 9.62E−08 | 6.94E−08 | 0.000010% | 0.000007% |
| 90 pulses, 4 Hz | 500 | 9.58E−05 | 7.09E−06 | 0.009582% | 0.000709% |
| 90 pulses, 4 Hz | 600 | 1.20E−02 | 3.17E−04 | 1.187906% | 0.031720% |
| 90 pulses, 4 Hz | 700 | 2.65E+00 | 2.47E−02 | 92.903098% | 2.444067% |
| 50 pulses, 4 Hz | 400 | 2.19E−07 | 6.20E−08 | 0.000022% | 0.000006% |
| 50 pulses, 4 Hz | 500 | 4.81E−06 | 4.43E−07 | 0.000481% | 0.000044% |
| 50 pulses, 4 Hz | 600 | 2.28E−04 | 6.81E−06 | 0.022814% | 0.000681% |
| 50 pulses, 4 Hz | 700 | 1.87E−02 | 1.79E−04 | 1.856629% | 0.017878% |
| 50 pulses, 4 Hz | 800 | 2.35E+00 | 7.11E−03 | 90.415357% | 0.708221% |
| 10 pulses, 10 Hz | 400 | 2.38E−08 | 1.10E−08 | 0.000002% | 0.000001% |
| 10 pulses, 10 Hz | 500 | 1.71E−07 | 1.41E−08 | 0.000017% | 0.000001% |
| 10 pulses, 10 Hz | 600 | 3.39E−06 | 2.15E−08 | 0.000339% | 0.000002% |
| 10 pulses, 10 Hz | 700 | 1.12E−04 | 4.48E−08 | 0.011233% | 0.000004% |
| 10 pulses, 10 Hz | 800 | 5.35E−03 | 1.39E−07 | 0.533480% | 0.000014% |
| 10 pulses, 10 Hz | 900 | 3.42E−01 | 6.34E−07 | 28.987215% | 0.000063% |
| 10 pulses, 10 Hz | 1000 | 2.76E+01 | 3.81E−06 | 100.000000% | 0.000381% |

It should be noted that parameter sets are explored with relative ease using a finite-element modeling approach, once model setup is accomplished, avoiding a need for undue experimentation to determine which parameters are preferable to avoid unacceptable thermal damage. Furthermore, in some embodiments, the number of pulses and/or other parameters are chosen based at least in part on the basis of experience with other electroporation protocol procedures and/or practical limitations of provided field generation equipment. For example, results of experiments such as those described in relation to FIGS. 8-10B show that heart muscle cells are ablated under exposure to field strengths of about 1000 V/cm, for 10 pulses of 100 μs at 1 Hz. Since voltage is normalized to field strength, these parameters are potentially independent of electrode design, and can be used in a model of electrode heating and electrical field distribution as a basis for selecting parameters of an electroporation protocol.

It is to be understood, for each electroporation protocol parameter, that one or more particular parameters can be set for the modeled conditions, while one or more others are selected based on modeled results under variable conditions. Moreover, it can be seen, from results shown in FIG. 6E and Table 2, that there are potentially a wide range of useful (volume-ablating and non-thermal) electroporation parameter settings available for use in particular embodiments of an electroporation protocol, with a particular electrode configuration. Other considerations can be applied to determine variations of a base protocol, without undue experimentation, for example, as described hereinbelow.

In some embodiments of the invention, a parameter set is selected based on meeting one or more criteria establishing a minimum peak electrical field strength within a volume of tissue targeted for ablation, for electroporation of tissue therein. In some embodiments, a parameter set is selected based on meeting one or more criteria establishing a maximum peak electrical field strength outside a volume of tissue targeted for ablation, for example to avoid excessive weakening and/or perforation of a tissue wall by electroporation beyond a targeted volume. Electrical field strengths potentially resulting in electroporation of tissue are described, for example, in relation to aspects of some embodiments of the invention hereinabove.

In some embodiments of the invention, a parameter set is selected based on a targeted total time for pulse delivery, for example, 5 seconds, 10 seconds, 15 seconds, or another longer, shorter, or intermediate pulse delivery time. A targeted pulse delivery time is itself selected, for example, to be short enough to avoid potentially dangerous ischemia in a patient due to interference with blood flow while an electrode array is deployed in a heart. It is a potential advantage to use a protocol which exceeds a targeted total time for pulse delivery by as little as possible, other parameters being equal. Total pulse delivery times are also described, for example, in relation to aspects of some embodiments of the invention hereinabove. In choosing a target time for pulse delivery, it should be understood that some interference with blood flow is expected for a period of several seconds up to a minute or more where the electrode catheter is being positioned, deployed, undeployed and/or withdrawn, in accordance, for example, with the experience of the field in the use of heart transcatheters in general.

In some embodiments of the invention, the duration of individual electroporating pulses is selected to be about the longest duration which is compatible (and/or compatible within some predetermined tolerance) with avoiding heating above an acceptable predicted threshold of thermal damage. In some embodiments of the invention, the threshold is, for example, thermal damage to 1% of molecules in a volume of tissue; or another threshold of thermal damage, for example as described hereinabove in relation to Table 2. In some embodiments of the invention, individual electroporating pulses are about, for example, 100 μsec long, 150 μsec long, or another pulse duration, for example as described in relation to aspects of some embodiments of the invention hereinabove. Potentially, a longer electroporating pulse makes it more likely that a membrane hole opened by the pulse will become irreversibly established. In some embodiments, later pulses are made shorter than early pulses. Potentially, this reduces thermal damage due to cumulative heating effects.

In some embodiments of the invention, the number of individual electroporating pulses is selected to be, for example, 10 pulses, or another number of pulses, for example as described in relation to aspects of some embodiments the invention hereinabove. In some embodiments of the invention, the number of pulses is selected to be the maximum deliverable within a targeted overall period of pulse delivery, without exceeding a threshold of thermal damage (other parameters being held equal, and optionally within some tolerance range). Optionally, pulse frequency is variable. For example, frequency is optionally lower late in the protocol, to help reduce cumulative thermal effects.

In some embodiments of the invention, information relating resistance (such as is calculated, for example, in _Table 2_) to expected thermal effects is tabulated for an electrode configuration. In some embodiments, resistance between electrodes of an electrode array is determined prior to and/or during an electroporation procedure protocol, and one or more parameters of the protocol varied on the basis of this measurement. In some embodiments, protocol changes are chosen (by calculation and/or lookup) to prevent ohmic heating from exceeding a threshold of thermal damage.

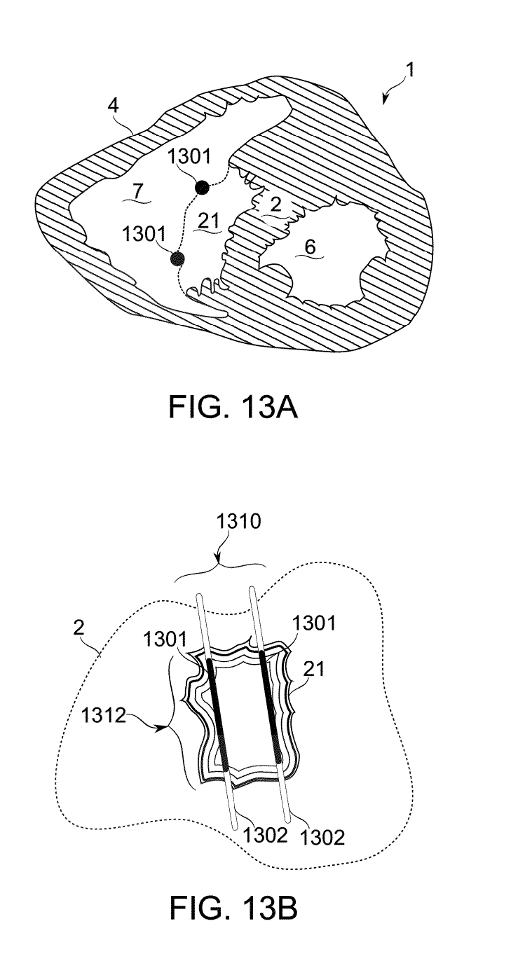

Reference is now made to FIGS. 13A-13B, which schematically illustrate electroporating ablation of a bulk 21 from a region of heart wall 2, according to some exemplary embodiments of the invention.

In some embodiments of the invention, electrodes 1301, illustrated in the transverse cross-section of FIG. 13A, are inserted into heart 1, and positioned against the ventricular septum 2 which separates the left ventricle 7 from the right ventricle 6. Alternatively or additionally the ventricular free wall 4 or another portion of heart wall is selected by placement of electrodes 1301.

Upon delivery of electroporating electrical fields through electrodes 1301, in some embodiments, a volume of tissue comprising tissue bulk 21 is initially inactivated. Over a period of time, typically a few days to a week, cellular material in tissue bulk 21 dissolves, causing a thinning of the septal wall 2 (or another targeted wall).

FIG. 13B illustrates a schematic view of an ablated heart wall 2, including electrodes 1301 and support member 1302 in an exemplary position for ablation, according to some embodiments of the invention. In some embodiments, the region ablated of tissue bulk 21 comprises a width 1310, a length 1312, and a depth. The extents of these dimensions are determined, for example, by parameters including electrical field strength, duration, period, and electrode positioning. A typical ablated tissue bulk measures, for example, about 4-12 mm wide by 12-24 mm long, with a depth of about 1-4 mm. Other exemplary widths, lengths, and depths of ablated tissue volumes are described hereinabove, for example, in connection with summary descriptions of aspects of some embodiments of the invention.

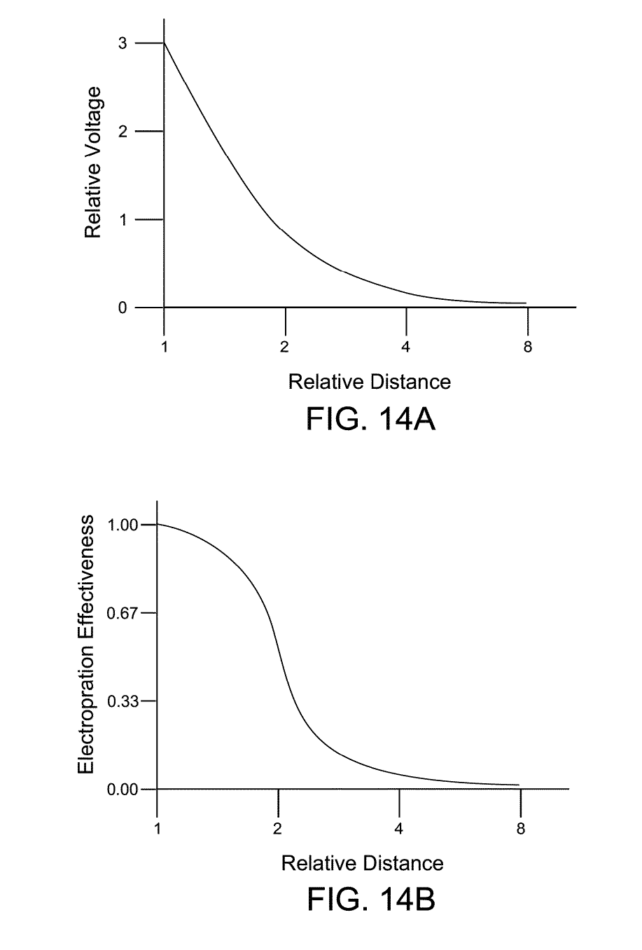

Reference is now made to FIGS. 14A-14B, which schematically illustrate non-linear relationships between distance, voltage field strength, and electroporation, according to some exemplary embodiments of the invention.

In some embodiments of the invention, as shown in FIG. 14A, relative voltage field strength falls off (at least in the limit of increasing distance) as an inverse squared function of distance from an electrode. It is to be understood that this rule is different in proximity to the electrodes, and in particular in the region in between electrodes, where the electrical field is more concentrated.

In some embodiments, and for example as schematically illustrated in FIG. 14B, cellular ablation due to electroporation is itself a non-linear function of the strength of the voltage field, with relative strengths of effects changing suddenly between changes in field strength. Potentially, cellular ablation as a function of electrode distance approximates a threshold and/or sigmoidal function. For example, review of the data of FIG. 6E shows that 10 electroporating pulses (for a particular cardiovascular target and electrode configuration) have an insignificant effect from 437.5 V/cm to 1750 V/cm. The interval from 1750 V/cm to 3500 V/cm, however, comprises most of the change in cellular loss due to changes in voltage. In an example from heart tissue, FIG. 9A shows a sharp transition between a region of thinned tissue 2201 and adjacent non-ablated regions.

Potentially, such non-linearities as a function of distance from electrodes allow approximately thresholded control over the boundary of a bulk of myocardial tissue targeted for electroporating ablation.

As used herein the term “about” refers to within ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

1. A method of reducing a volume of myocardial tissue in a mammalian heart by exposure to an electrical field, comprising:
positioning an electrode array comprising at least a current source electrode and a current sink electrode to select a target bulk comprising said myocardial tissue; and
delivering a pulsed electrical field through said electrode array to said target bulk, electroporating cells therein in a continuous volume extending between said current source electrode and said current sink electrode;
said electroporation of cells leading to reduced volume of myocardial tissue in said target bulk.

2. The method of claim 1, wherein said reducing comprises death of cells within said bulk of myocardial tissue.

3. The method of claim 2, wherein said death of cells within said bulk of myocardial tissue comprises death after irreversible electroporation of cellular membranes.

4. The method of claim 1, wherein thermal heating due to said pulsed electrical field is below a threshold of thermal damage to the intracellular matrix within said target bulk.

5. The method of claim 4, wherein said threshold of thermal damage is 55° C. or lower.

6-7. (canceled)

8. The method of claim 1, wherein said target bulk comprises a portion of the wall of the left ventricle of said heart.

9. The method of claim 1, wherein said target bulk extends over from between 1-9 cm² of a wall region of said heart, to a depth within said tissue of at least 1 mm.

10. The method of claim 1, wherein said pulsed electrical field comprises a peak field strength above 250 V/cm extending continuously between said source electrode and said sink electrode.

11. The method of claim 1, wherein said pulsed electrical field is delivered in pulses sufficiently short to remain below a threshold of thermal damage to said myocardial tissue.

12. (canceled)

13. The method of claim 10, wherein said pulsed electrical field is delivered in pulses at a sufficient interval to avoid cumulative thermal buildup to a threshold of thermal damage.

14. (canceled)

15. The method of claim 1, wherein said electrode array comprises at least three electrodes.

16. The method of claim 15, wherein electrodes of said electrode array are activated at least partially asynchronously during said delivery of said pulsed electrical field.

17-21. (canceled)

22. An apparatus for reducing a volume of myocardial tissue in the wall of a mammalian heart, comprising:
a plurality of electrodes comprising a current source electrode and a current sink electrode;
said plurality of electrodes being disposed on the distal end of a catheter and insertable to said heart thereby;
a voltage source, configured to deliver a predetermined electrical potential to said plurality of electrodes when deployed in said heart; and
said plurality of electrodes being deployable within said heart to assume positions against said wall and predetermined relative to each other; wherein
said deployed positions define a volume by the electrical field produced upon delivery of said electrical potential, said volume extending continuously between said current source electrode and said current sink electrode, and being comprised in a bulk of myocardial tissue, said myocardial tissue being comprised in said wall, which said bulk being irreversibly ablated by electroporation upon delivery of one or more pulses of said electrical potential.

23-32. (canceled)

33. The apparatus of claim 22, comprising a frame expandable to urge said plurality of electrodes toward said bulk of myocardial tissue, and to distance them to predetermined relative positions, while said distal end of said catheter is inserted into said heart.

34. (canceled)

35. The apparatus of claim 22, wherein said electrical field within said bulk of myocardial tissue comprises a field region having a maximum field strength above 250 V/cm during a period when said electrical potential is received.

36. The apparatus of claim 35, wherein said field region has everywhere within said bulk of myocardial tissue a maximum field strength above 250 V/cm during said period.

37-41. (canceled)

42. The apparatus of claim 22, wherein said plurality of electrodes comprises at least three electrodes.

43. The apparatus of claim 42, comprising a switching mechanism for directing the application of voltage potential to said plurality of electrodes, configured such that said at least three electrodes are actuatable to receive said electrical potential at least partially asynchronously from one another.

44. The apparatus of claim 42, wherein at least a portion of said bulk of myocardial tissue is subjectable to an electroporating electrical field delivered from at least two sets of electrodes differing in at least one member during a period when said electrical potential is received.

45. (canceled)

46. The apparatus of claim 22, comprising a thermal sensor disposed near the position of at least one electrode of said plurality of electrodes.

47-50. (canceled)
